(19) United States
(12) Patent Application Publication
(10) Pub. No.: US 2026/0092085 A1
(43) Pub. Date: Apr. 2, 2026

(54) SYNTHETIC TRIPLEX PEPTIDE NUCLEIC ACID-BASED INHIBITORS FOR CANCER THERAPY

(71) Applicants: University of Connecticut, Farmington, CT (US); Beth Israel Deaconess Medical Center, Inc., Boston, MA (US)

(72) Inventors: Raman Bahal, Glastonbury, CT (US); Karishma Dhuri, Mansfield Center, CT (US); Frank Slack, Waban, MA (US)

(21) Appl. No.: 18/858,646

(22) PCT Filed: Apr. 20, 2023
(86) PCT No.: PCT/US2023/066006

§ 371 Date: Oct. 21, 2024

Related U.S. Application Data
(60) Provisional application No. 63/334,787, filed on Apr. 26, 2022.

(51) Int. Cl. C07K 14/00; A61K 38/00; A61P 35/00

(52) U.S. Cl. CPC C07K 14/003; A61P 35/00; A61K 38/00

(57) ABSTRACT

A novel peptide nucleic acid (PNA) oligomer capable of forming a PNA/RNA/PNA triplex when binding to its target RNA is described. An anti-micro RNA (miRNA) capable of binding miR-155 was designed based on the novel PNA oligomer and was shown to significantly decrease miR-155 expression in vitro in lymphoma cell lines. In vivo testing in xenograft mouse models resulted in reduced miR-155 expression followed by reduced tumor growth. Methods of making and using the novel PNA oligomer for targeting other coding and noncoding RNAs are described.

### CROSS-REFERENCE TO RELATED APPLICATIONS

This application is a National Stage Application of International Patent Application No. PCT/US23/66006, filed 20 Apr. 2023, which claims priority to, and the benefit of, U.S. Provisional Application 63/334,787, filed on Apr. 26, 2022, each of which is incorporated by reference herein in its entirety.

### STATEMENT REGARDING FEDERALLY SPONSORED RESEARCH & DEVELOPMENT

This invention was made with government support under CA241194 awarded by the National Institutes of Health. The government has certain rights in the invention.

### SEQUENCE LISTING

The Instant Application contains a Sequence Listing which has been submitted electronically in XML format and is hereby incorporated by reference in its entirety. Said XML copy, created on 27 May 2025, is named “UCT0289US2_Sequence_Listing.xml” and is 33,622 bytes in size.

### BACKGROUND

MicroRNA (miRNA or miR) is a class of non-coding RNAs that control gene expression at the post-transcription level. miRNAs play key roles in maintaining physiological processes by controlling gene expression through regulating messenger RNA (mRNA) stability and translation. Aberrant expression of miRNAs causes several devastating diseases. In cancer, atypical miRNA levels lead to altered processes, including differentiation, proliferation, and apoptosis. miRNAs have been explored as promising molecular targets for the development of precision medicine in cancer.

Synthetic nucleic acid-based antimiRs have been evaluated in conjunction with delivery systems to repress miRNAs upregulated in multiple tumors (also called oncomiRs) for potential cancer therapeutics. Several antimiRs have been developed to specifically target full-length miRNAs by Watson-Crick recognition to prevent their interaction with target mRNAs. In particular, peptide nucleic acids (PNAs) have gained attention as potential antimiR agents. PNAs are synthetic nucleic acid analogs that possess a neutral backbone and are resistant to enzymatic degradation. It is well-known that PNAs can target the full-length miRNAs by Watson-Crick base pairing and thus control gene expression. Targeting full-length miRNAs provides numerous advantages, especially the sequence-specific targeting of preferred miRNA sites minimizes the off-target toxicity. A few studies reported using shorter antimiRs targeting the miRNA seed region to inhibit its function. Still, clinical translation of targeting seed region-based strategies could be hampered due to off-target effects because of non-specific binding with other coding and non-coding RNAs. Though promising results have been shown, increasing the binding affinity of antimiRs without compromising their specificity remains a continuous goal.

### BRIEF SUMMARY

In an aspect, a peptide nucleic acid (PNA) oligomer forms a PNA/RNA/PNA triplex structure, wherein the PNA oligomer has the formula:

5′-first PNA segment-flexible linker-second PNA segment-3′
wherein the first PNA segment is complementary to a homopurine stretch in the RNA,
the second PNA segment is complementary to a region of the RNA including the homopurine stretch,
wherein the first PNA segment and the second PNA segment form the PNA/RNA/PNA triplex structure with the RNA,
and wherein the RNA is a coding or noncoding RNA.

In another aspect, a method for reducing expression of a targeted RNA involved in health disorders in a subject comprises providing to a cell of the subject in vivo or ex vivo a PNA oligomer, wherein the binding of the PNA oligomer to the targeted RNA reduces expression of the targeted RNA.

### BRIEF DESCRIPTION OF THE DRAWINGS

FIGS. 1A-D show design of PNA and gamma PNA-155 oligomers and gel shift binding assay.

(1A) Chemical structure of PNA and serine-γPNA units. B signifies nucleobases Adenine (A), Guanine (G), Cytosine (C) and Thymine (T).

(1B) The left panel is a schematic of conventional full length PNA-155 X₁ACCCCTATCACGATTAGCATTAA X₁, wherein X₁ is RR; SEQ ID NO: 1) binding with the target miR-155 (UUAAUGCUAAUCGUGAUAGGGGU (SEQ ID NO: 2)). PNA-155 binds by Watson-Crick base pairing, for example. In the right panel gtcPNA-155 (X₁X₂ACCCCTATCACGATTAGCATTAAX₁, wherein X¹ is RR wherein X2 is TJJJJ-linker-; SEQ ID NO: 3) binds by Watson-Crick and Hoogsteen binding domain. J signifies pseudoisocytosine nucleobase. Linker (11-Amino-3,6,9-Trioxaundecanoic Acid, DCHA) is represented as —OOO—.

(1C) The oligomer sequences of PNA-155 and γPNA-155 (both the standard PNA and the γ-PNA have the same sequence, SEQ ID NO: 1) and γtcPNA-155 (SEQ ID NO: 3 with and without a TAMARA label) are designed to bind to the full length of target miR-155. Scramble PNA (Scr-γtcPNA-155) was synthesized as a control (X₁X₃ACX₄TGCCATTX₄CACGAACX₄CTX₁, X₁ is RR, X₃ is ATJTA-linker, wherein J is pseudoisocytosine and the linker is 11-Amino-3,6,9-Trioxaundecanoic Acid, DCHA) represented as —OOO—, X₄ is pseudoisocytosine, SEQ ID NO: 4). TAMRA (5-Carboxytetramethylrhodamine) appended to γtcPNA-155. The five PNAs have two arginine (R) residues on each N- and C-terminus ends.

(1D) Dose-dependent gel shift binding assay of target miR-155 (1 mM) with PNA-155 and γtcPNA-155 at indicated concentrations. The samples were prepared in the physiological buffer (2 mM MgCl₂, 150 mM KCl, 10 mM NaPi) and incubated for 24 hours at physiological temperature (37° C.) followed by PAGE separation and visualization of bands by SYBr® Gold staining. Inset number shows different mode of binding (i) unbound miR-155 target (ii) PNA-155 binding with the target miR-155 by Watson-Crick domain. (iii) γtcPNA-155 binding with the target miR-155 by Watson Crick and Hoogsteen base pairing. (iv) γtcPNA-155 binding with the miR-155 by Watson-Crick base pairing. (v) Clamp segment of γtcPNA-155 binding with the target miR-155 by Hoogsteen base pairing.

FIG. 2 shows RP-HPLC profiles of synthesized PNA-155, γtcPNA-155, Scr-γtcPNA-155 and γPNA-155.

FIGS. 3A-F show cell culture-based functional assay in U2932 lymphoma cells. (3A) γtcPNA-155-TAMRA uptake in the U2932 lymphoma cell line: Representative flow cytometry traces of TAMRA fluorescence in U2932 cells after treatment with 500 nM dose of γtcPNA-155-TAMRA for 48 hours. The data was analyzed by FlowJo software. (3B) Normalized miR-155 gene expression levels in U2932 after treatment with phosphate buffer saline (PBS) as a control and with 500 nM dose of Scr-γtcPNA-155, PNA-155, γPNA-155 and γtcPNA-155 for 48 hours compared to average control U6 (n=3), data represented as mean±standard error mean (SEM), Statistical analysis was performed using Unpaired two-tailed t-test. Further, statistical analysis was performed relative to Scr-γtcPNA-155 treated cells. ***p<0.001. (3C) Gene expression level of miR-155 downstream genes, tumor suppressor proteins (FOXO3A, CUX1, SOCS1, CSF1R, JARID2, SHIP1, PICALM, PDCD4, BACH1, WEE1, TP53TG3, CASP3, PTEN) in U2932 cell line after treatment with PBS as a control and with after treatment with 500 nM of Scr-γtcPNA-155, PNA-155, γPNA-155 and γtcPNA-155 for 48 hours. Data is normalized with average GAPDH control (n=3), and represented as mean±SEM, *p<0.05. Unpaired two-tailed t-test was used for statistical analysis and analysis was performed relative to Scr-γtcPNA-155 treated cells. (3D) Gene expression level of miR-155 downstream genes MCL1 in U2932 cell line after treatment with PBS as a control and 500 nM of Scr-γtcPNA-155, PNA-155, γPNA-155 and γtcPNA-155 for 48 hours. Data is normalized with average GAPDH control (n=3), and represented as mean±SEM, *p<0.05, **p<0.01 and ***p<0.001. Statistical analysis was performed using Unpaired two-tailed t-test. Analysis was performed relative to Scr-γtcPNA-155 treated cells. (3E) Representative western blot of Mcl-1 and its quantification (n=3 technical replicate) and (3F) Caspase-3 protein and its quantification (n=3 technical replicate) in U2932 cell line after treatment with 500 nM of Scr-γtcPNA-155, PNA-155 and γtcPNA-155 for 48 hours. Data is represented as mean±SEM and unpaired two-tailed t-test was used for statistical analysis. *p<0.05 **p<0.01. Numbers above western blot panels represent relative quantification of the respective bands using ImageJ software and normalized relative to loading control and treatment control.

FIGS. 4A-B show TAMRA fluorescence. (4A) Representative flow cytometry trace in SUDHL-2 cells after treatment with 500 nM dose of γtcPNA-155-TAMRA for 48 hours. (4B) Cellular uptake studies by confocal microscopy in U2932 cell line after treatment with γtcPNA-155-TAMRA. Scale bar represents 30 μm.

FIGS. 5A-B show representative western blot of Mcl-1 (5A) and Caspase-3 (5B) and its quantification (n=3) obtained from U2932 cell line after treatment with 500 nM of Scr-γtcPNA-155, γPNA-155 and γtcPNA-155 for 48 hours. Data is represented as a mean±SEM and unpaired t-test was used for statistical analysis. *p<0.05. The relative protein levels were determined from the band intensity using ImageJ software and normalized relative to loading control and treatment control.

FIG. 6 shows normalized miR-155 gene expression levels in SUDHL-2 cells after treatment with 500 nM dose of Scr-γtcPNA-155, PNA-155, and γtcPNA-155 for 48 hours compared to average control U6 (n=3). Data represented as mean±standard error mean (SEM). Unpaired two-tailed t-test was used for statistical analysis, **p<0.01. The analysis was performed relative to Scr-γtcPNA-155.

FIG. 7 shows gene expression level of certain validated miR-155 downstream genes, tumor suppressor proteins (FOXO3A, CUX1, SOCS1, JARID2, SHIP1, PICALM, PDCD4, BACH1, WEE1, TP53TG3, CASP3, PTEN) in SUDHL-2 cell line after treatment with 500 nM of Scr-gtcPNA-155, PNA-155 and gtcPNA-155 for 48 hours. Data is normalized with average GAPDH control (n=3), and represented as mean±SEM, **p<0.01. Unpaired two tailed t-test was used for statistical analysis and the analysis was performed relative to Scr-gtcPNA-155.

FIG. 8 shows dose-dependent cell viability in U2932, SUDHL-2, and SUDHL-5 cells after treatment with Scr-γtcPNA-155, PNA-155 and γtcPNA-155 for 48 hours. Cell viability was performed using trypan blue-based assay (n=3 technical triplicate), data represented as mean±(SEM), * represents the statistical analysis were performed relative to Scr-γtcPNA-155 and ♦ represent analysis performed relative to PNA-155. *p<0.05, **p<0.01, ***p<0.001 and ****p<0.0001, ♦p<0.05, ♦♦p<0.01, unpaired two-tailed t-test was used for statistical analysis. The experiment was repeated three times and in triplicate.

FIG. 9 shows cell viability in U2932, SUDHL-5 and SUDHL-2 cells after treatment with 500 nM of Scr-γtcPNA-155, PNA-155, γPNA-155 and γtcPNA-155 for 48 hours. Cell viability was performed using trypan blue-based assay (n=3), data represented as mean±standard error mean (SEM), *p<0.05, **p<0.01. Unpaired two-tailed t-test was used for statistical analysis.

FIG. 10 shows dose-dependent cell viability in U2932, SUDHL-2, and SUDHL-5 cells after treatment with Scr-γtcPNA-155 for 48 hours. Cell viability was performed using trypan blue-based assay (n=3), data represented as mean±standard error mean (SEM).

FIG. 11 shows quantification of apoptosis by Annexin-based assay. Quantification of apoptotic cells by flow cytometry after treating U2932 cells with PBS as a control and 500 nM Scr-γtcPNA-155, PNA-155, γPNA-155 and γtcPNA-155 for 48 hours. The apoptotic cells and necrotic were stained using Phycoerythrin (PE) Annexin V and 7-Amino-Actinomycin (7-AAD) respectively. The % apoptotic cells after treatment was compared to the PBS treated cells, by setting the same threshold. Bar graph of % apoptotic cells (n=3 technical triplicate), data represented as mean±standard error mean (SEM), *p<0.05, **p<0.01, ***p<0.001, unpaired two-tailed t-test was used for statistical analysis. Representative dot plots of Scr-γtcPNA-155 and γtcPNA-155 treated U2932 cells. The experiment was repeated three times and in triplicate. One out of three representative experiments are shown.

FIG. 12 shows confocal fluorescent images of Annexin V-FITC in U2932 cells treated with 500 nM Scr-γtcPNA-155, PNA-155, γPNA-155 and γtcPNA-155 for 48 hours. Scale bar is 100 μm.

FIG. 13 shows quantification of apoptotic cells by flow cytometry after treating U2932 cells with PBS as a control and 10UM γtcPNA-155 for 48 hours. The apoptotic cells and necrotic were stained using Phycoerythrin (PE) Annexin V and 7-Amino-Actinomycin (7-AAD) respectively. The % apoptotic cells after treatment was compared to the PBS treated cells, by setting the same threshold. Bar graph of % apoptotic cells (n=3 technical triplicate), data represented as mean±standard error mean (SEM), **p<0.01, unpaired two-tailed t-test was used for statistical analysis. Representative dot plots of PBS treated and γtcPNA-155 treated U2932 cells. The experiment was repeated three times and in triplicate. One out of three representative experiments is shown.

FIG. 14 shows safety assessment of PNA-155 and γtcPNA-155 treatment at indicated doses for 48 hours in PBMC cells by trypan blue assay. Control is PBS treated PBMC cells. n=3, data represented as mean±standard error mean (SEM).

FIG. 15 shows workflow for the in vivo treatment in the NSG mice model containing U2932 subcutaneous tumors. I.T indicates the injection are given intratumorally.

FIG. 16 shows localization of γtcPNA-155 TAMRA in U2932 tumors after intratumoral injection. Inset shows the IVIS imaging of tumors containing γtcPNA-155-TAMRA.

FIGS. 17A-D show in vivo studies in U2932 derived xenograft model. (17A) Biodistribution of γtcPNA-155 TAMRA in tumor sections (17B) Tumor volume fold change (n=6, data represented as mean±standard error mean (SEM), *p<0.05, **p<0.01, multiple t-tests one per row was used for statistical analysis). Asterisk denotes the analysis was performed relative to Scr-γtcPNA-155. Diamond symbol denotes the analysis was performed relative to PNA-155. (17C) Representative images showing the effects of Scr-γtcPNA-155, PNA-155, γPNA-155 and γtcPNA-155 on Caspase-3 immunostaining of tumor (magnification; ×20). Autofluorescence (AF) at 514 nm was used to show the tumor. Scale bar=50 mm. (17D) Caspase-3 immunostaining quantification, *p<0.05. n=15. The n indicates the number of images.

FIG. 18 shows organ weights of mice treated with Scr-γtcPNA-155, PNA-155, γPNA-155 and γtcPNA-155. n=3, data represented as mean±standard error mean (SEM).

FIG. 19 shows representative hematoxylin and eosin-stained images showing the effects of γtcPNA-155 on the histology of liver and kidney (magnification ×20).

FIG. 20 shows representative U2932 tumor images of the PBS, PNA-155 and γtcPNA-155 treated mice. The corresponding resected tumor harvested after sacrificing the mice on the nineth day of the treatment.

FIGS. 21A-B show Ki-67 immunostaining (21A) and quantification (21B) in the in vivo treated tumors (n=15), **p<0.01. The n indicates the number of images. Scale bar=50 μm.

FIGS. 22A-D show in vivo gene expression and protein level analysis. (22A) miR-155 gene expression levels in U2932 tumor samples after in vivo treatment with indicated PNAs. Data was normalized with average control U6 (n=4, data represented as mean±standard error mean). ***p<0.001 (22B) Gene expression level of downstream target genes of miR-155: MCL1 in U2932 tumor samples after treatment with total 3 mg kg-1 dose of Scr-γtcPNA-155, PNA-155, γPNA-155 and γtcPNA-155 and normalized with average control GAPDH (n=3, data represented as mean±SEM), unpaired two-tailed t-test was performed for analysis). *p<0.05, **p<0.01 (22C) Representative western blot of Mcl-1 protein and its quantification (n=3 technical replicate) in U2932 tumor samples treated with total 3 mg kg-1 dose of Scr-γtcPNA-155, PNA-155 and γtcPNA-155. Data is represented as mean±SEM and unpaired two-tailed t-test was performed for analysis. *p<0.05. The relative protein levels were determined from the band intensity using ImageJ software and normalized relative to loading control and treatment control. (22D) Gene expression level of tumor suppressor proteins (FOXO3A, CUX1, SOCS1, CSF1R, JARID2, SHIP1, PICALM, PDCD4, BACH1, CASP3) in U2932 tumor samples after treatment with total 3 mg kg-1 dose of Scr-γtcPNA-155, PNA-155, γPNA-155 and γtcPNA-155 and normalized with average control GAPDH (n=3, data represented as mean±SEM, unpaired two-tailed t-test was performed for statistical analysis). *p<0.05, **p<0.01.

FIG. 23 shows representative western blot of Mcl-1 protein and its quantification (n=3) obtained from U2932 tumor xenograft after treatment with Scr-γtcPNA-155, γPNA-155 and γtcPNA-155. Data is represented as mean±SEM and unpaired t-test was used for statistical analysis *p<0.05. The relative protein levels were determined from the band intensity using ImageJ software and normalized relative to loading control and treatment control.

FIG. 24 shows representative western blot of Mcl-1 protein and its quantification (n=3) obtained from U2932 xenograft tumor after treatment with Scr-γtcPNA-155. Data is represented as mean±SEM and unpaired t-test was used for statistical analysis.

FIG. 25 shows representative western blot of Caspase-3 protein and its quantification (n=3) obtained from U2932 tumor xenograft after treatment with Scr-γtcPNA-155, PNA-155 and γtcPNA-155. Data is represented as mean±SEM and unpaired t-test was used for statistical analysis. The relative protein levels were determined from the band intensity using ImageJ software and normalized relative to loading control and treatment control.

FIG. 26 shows tumor volume fold change after intratumoral treatment of Scr-γtcPNA-155, PNA-155 and γtcPNA-155 in SUDHL-2 cell line derived xenograft. Data represented as mean±standard error mean (SEM). Scr-γtcPNA-155 (n=4), PNA-155 (n=5), γtcPNA-155 (n=5), *p<0.05, **p<0.01, ***p<0.001, ****p<0.0001. Multiple t-tests one per row was used for statistical analysis.

FIG. 27 shows miR-155 gene expression analysis in the tumors from SUDHL-2 cell line derived xenograft after treatment with indicated PNAs. Data normalized with average control U6 (n=4) and represented as mean±standard error mean. Unpaired t test was used for statistical analysis. Further statistical analysis was performed relative to Scr-γtcPNA-155. **p<0.01, ***p<0.001.

The above-described and other features will be appreciated and understood by those skilled in the art from the following detailed description, drawings, and appended claims.

### DETAILED DESCRIPTION

Described herein are novel chemically modified peptide nucleic acid (PNA) oligomer antiRNAs capable of binding to RNA and forming a PNA/RNA/PNA triplex. Particularly when the PNAs are serine gamma modified, the gPNAs target RNA more efficiently compared to the conventional full length PNAs based on their binding affinity. Adding a tail-clamp to the γPNA, γtcPNA, not only improved its affinity toward the target RNA but also increased its efficacy in retarding disease.

Diffuse Large B-Cell Lymphoma (DLBCL) is an aggressive lymphoma that can arise in lymph nodes or outside of the lymphatic system, in the gastrointestinal tract, testes, thyroid, skin, breast, bone or brain. A combination of chemotherapy and a monoclonal antibody targeting CD20 remains the backbone of most treatments. The most widely used treatment for DLBCL is R CHOP (rituximab, cyclophosphamide, doxorubicin, vincristine, and prednisone) that is usually given in 21-day cycles. miR-155 is highly dysregulated in DLBCL and is therefore an important molecular target for developing precision medicine for lymphoma therapy.

The novel PNA oligomers described herein were tested by designing anti-miR-155 PNAs targeting microRNA-155 (miR-155) in the diffuse large B cell lymphoma (DLBCL) disease model. The anti-miR-155 PNAs were comprehensively tested in multiple lymphoma cell lines and results corroborated by gene expression, western blot analysis, and cell viability-based functional studies, in addition to preclinical testing in vivo in xenograft mouse models. Results showed a significant decrease in miR-155 expression followed by reduced tumor growth in the in vivo treated group supporting a therapeutic application of this novel strategy. Gamma-modified tail-clamp PNA-based molecules can be used to target miRNA, mRNAs and other non-coding RNAs involved in the development of health disorders.

In an aspect, a peptide nucleic acid (PNA) oligomer that forms a PNA/RNA/PNA triplex structure, where the RNA is a target RNA, and where the PNA oligomer has the formula: 5′-first PNA segment-flexible linker-second PNA segment-3′ wherein the first PNA segment is complementary to a homopurine stretch in the target RNA, the second PNA segment is complementary to a region of the target RNA including the homopurine stretch, wherein the first PNA segment and the second PNA segment form the PNA/RNA/PNA triplex structure with the RNA, and wherein the RNA is a coding or noncoding RNA. In an aspect, the first PNA segment is a tail clamp. In another aspect, one or both, preferably both, PNA segments are gamma-modified, wherein the PNA backbone contains one or more modification in the gamma-position of the N-(2-aminoethyl)glycine unit. Gamma modified PNA can improve solubility, reduce self-aggregation, and/or result in more stable PNA-RNA hybrids. Gamma PNA modifications include serine modified, lysine modified, glutamic acid modified, or alanine modified. In an aspect, the first, second or both PNA segments are serine gamma modified.

As used herein, PNA is a synthetic form of a nucleic acid which lacks a net electrical charge along its protein-like backbone. Specifically, PNAs are molecules in which the phosphate backbone of an oligonucleotides is replaced in its entirety by repeating N-(2-aminoethyl)-glycine units and phosphodiester bonds are replaced by peptide bonds. The various heterocyclic bases are linked to the backbone by methylene carbonyl bonds. PNAs maintain spacing of heterocyclic bases that are similar to oligonucleotides but are achiral and neutrally charged molecules. PNAs are comprised of peptide nucleic acid monomers. The heterocyclic bases can be any of the standard bases (uracil, thymine, cytosine, adenine and guanine) or any of the modified heterocyclic bases described below.

PNAs, typically single stranded, can bind to a target nucleic acid, e.g., RNA or DNA, via Watson-Crick hydrogen bonds, but with binding affinities significantly higher than those of a corresponding oligonucleotide composed of DNA or RNA. The neutral backbone of PNAs decreases electrostatic repulsion between the PNA and target RNA phosphates. As used herein, the PNA binds RNA sufficiently to prevent expression of the bound RNA.

As used herein, the PNA oligomer comprises two PNA molecules linked together by a linker of sufficient flexibility to form a single PNA molecule which forms the PNA/RNA/PNA triplex structure with the RNA. An exemplary linker is between 1 and 10 units of 8-amino-3,6-dioxaoctanoic acid, referred to as an O-linker, and 6-aminohexanoic acid, 8-amino-2, 6, 10-trioxaoctanoic acid, or 11-amino-3, 6, 9-trioxaundecanoic acid. Poly(ethylene) glycol monomers can also be used as PNA linkers. A PNA linker can contain multiple linker molecule monomers in any combination.

The first PNA segment in the PNA oligomer is a pyrimidine stretch that hybridizes to a homopurine stretch on the target RNA, also referred to as a “tail” or tail clamp (tc) added to the end of the Watson-Crick binding portion. The tail clamp binds portions of the target nucleic acid or RNA by Hoogsteen base-pairing. The PNA oligomer with the tail clamp (tcPNA) mediates a mode of binding to RNA that encompasses both triplex and duplex formation with the tail clamp PNA forming a triplex portion, the PNA/RNA/PNA triplex, in addition to the second segment's PNA/RNA duplex portion.

Preferably, both the Watson-Crick and Hoogsteen binding portions of the triplex forming molecules are substantially complementary to the target sequence. In some aspects, the Hoogsteen binding segment of the PNA oligomer includes one or more, chemically modified cytosines such as pseudocytosine, pseudoisocytosine, and 5-methylcytosine.

In an aspect, the first RNA segment comprises one more pseudoisocytosine units. In another aspect, the first RNA segment comprises only pseudoisocytosine units and thymidine units.

The second segment of the PNA oligomer is complementary to a region of the RNA including the homopurine stretch. Specifically, it forms Watson-Crick bonding with the target RNA to selectively bind to or hybridize with a predetermined target sequence, target region, or target site within an RNA such that a triple-stranded structure is formed. The nucleotide sequence of the second PNA oligomer segment is selected based on the sequence of the target sequence, the physical constraints to achieve binding of the oligonucleotide within the major groove of the target region, and preferably to have a low dissociation constant (Kd) for the oligonucleotide/target sequence.

In some embodiments, the PNA oligomer including a first Hoogsteen binding peptide nucleic acid (PNA) segment and a second Watson-Crick binding PNA segment collectively total no more than 50 nucleobases in length.

In an aspect, depending on the length of the target RNA, the second PNA segment can be the full length or partial length of the target RNA. In one aspect, the PNA is about 7-10 nucleotides in length, about 5-12 nucleotides in length, about 7-15 nucleotides in length, about 10-20 nucleotides in length, about 7-30 nucleotides in length, and up to the full length of the target RNA.

In an aspect the first and second segments of the PNA oligomer bind to or hybridize to the target sequence under conditions of high stringency and specificity. Most preferably, the oligomers bind in a sequence-specific manner to the target RNA. Reaction conditions for in vitro triple helix formation of an PNA oligomer to a nucleic acid sequence vary from oligomer to oligomer, depending on factors such as oligomer length, the number of G: C and A: T base pairs, and the composition of the buffer utilized in the hybridization reaction. An oligomer substantially complementary to the target region of the nucleic acid molecule is preferred.

The PNA oligomers can also include other positively charged moieties to increase the solubility of the PNA, for increased cell permeability, and/or to increase the affinity of the PNA for the target RNA. Commonly used positively charged moieties include the amino acids lysine and arginine, although other positively charged moieties may also be useful. Lysine and arginine residues can be added to a tcPNA linker or can be added to the carboxy or the N-terminus of a PNA oligomer strand.

Exemplary modifications to PNA include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to RNA to generate a chimeric oligomer, redesign of the backbone architecture, conjugation of PNA to DNA or RNA. These modifications improve solubility but often result in reduced binding affinity and/or sequence specificity.

In an aspect, PNA is synthesized using monomers by established solid-phase synthesis based protocols known in the art.

In some aspects, one or more PNA monomers forming a PNA oligomer are modified at the gamma position in the polyamide backbone (γPNAs) as illustrated below (wherein “B” is a nucleobase and “R” is a substitution at the gamma position).

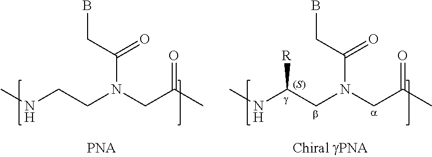

Substitution at the gamma position creates chirality and provides helical pre-organization to the PNA oligomer, yielding substantially increased binding affinity to the target RNA. Other advantageous properties can be conferred depending on the chemical nature of the specific substitution at the gamma position (the “R” group in the chiral γPNA above). The synthesis of γPNAs is described in U.S. Pat. No. 10,221,216, incorporated herein by reference for the disclosure of γPNA and methods of synthesis of γPNA.

Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

In one aspect, the PNA oligomer forming a PNA/RNA/PNA triplex is a γPNA with a tail clamp, or a γtcPNA.

Chemical modifications of the basic PNA structure are known and can be used. For example, fluorine-modified, cyclopentyl-modified, mini-peg-modified, guanidinium-modified, pyrrolidinyl-modified, and 2-aminopyridine-modified PNAs are known in the art and can be chosen for preparation of the PNA oligomer to improve cell permeability or increase RNA binding affinity.

Mini-Peg-containing γ-PNAs are described in U.S. Pat. No. 10,793,605, incorporated herein by reference for its disclosure of mini-PEG γ-PNAs and their methods of synthesis.

In another aspect, provided herein is a method of reducing expression of a targeted RNA involved in health disorders in a subject, comprising providing to a cell of the subject in vivo or ex vivo a PNA oligomer capable of forming a PNA/RNA/PNA triplex with the targeted RNA, wherein the binding of the PNA oligomer to the targeted RNA reduces expression of the targeted RNA. Preferably, the targeted RNA contains a 5′ purine stretch.

In an aspect, the PNA modifies the expression of coding or noncoding RNA in a cell. RNAs carrying the code for protein synthesis are “coding RNA”. Noncoding RNAs do not undergo translation to synthesize proteins but may have gene regulation functions, both in normal or disease cells. Noncoding RNAs include ribosomal RNA, transfer RNA (about 89 nucleotides), small nuclear RNA (snRNA; about 150 nucleotides), small nucleolar RNA (snoRNA; about 60-300 nucleotides), Piwi-interacting RNAs (piRNA; about 24-30 nucleotides), microRNA (miRNA; about 18-25 nucleotides), and long noncoding RNA (lncRNA; larger than 200 nucleotides in size).

As exemplified herein below, expression of a microRNA, mi-R155 was inhibited or reduced using the methods described herein. MicroRNAs (miRNAs) are noncoding RNAs that play important roles in regulating gene expression by activating translation or transcription, in most cases by interacting with mRNA at the 3′ or 5′untranslated region to induce mRNA degradation and translational repression. MicroRNAs have been demonstrated to play a major role in a wide range of developmental processes including metabolism, cell proliferation, apoptosis, developmental timing, neuronal cell fate, among others.

In some aspects, the PNA oligomer can have a sequence substantially complimentary to a target miRNA, or an anti-miR, such that it allows formation of the triplex with the target miRNA. The second PNA segment can be designed to be complementary to the full length or partial length of the target miRNA. Accordingly, in some embodiments, anti-miR-155 PNA oligomer can comprise the full miR-155 sequence 5′ACCCCTATCACGATTAGCATTAA 3′ (SEQ ID NO:5), or a portion or variant thereof such as 5′CCCCTATCACGATTAGCATTAA3′ (SEQ ID NO: 6) that retains the ability to bind to miR-155. In another embodiment, the PNA oligomer includes a tail clamp, tcPNA-anti-miR-155 having the sequence 5′ X₂CCCCTATCACGATTAGCATTAA3′, X₂ is TJJJJ-linker, wherein J is pseudoisocytosine and the linker is 11-Amino-3,6,9-Trioxaundecanoic Acid, DCHA) represented as —OOO— (SEQ ID NO:7), or a portion or variant thereof that retains the ability to bind to miR-155. Bases for facilitating entry into the cell can be added on the carboxy or amino terminus, or both, of the anti-miR-155 PNA oligomer. Therefore, an anti-MiR-155 oligomer can comprise the full miR-155 sequence with, for example additional carboxy or amino terminus arginine residues (SEQ ID NO:1). In another aspect, a tcPNA-anti-miR-155 oligomer can have the sequence (SEQ ID NO:3), or a portion or variant thereof that retains the ability to bind to miR-155.

Aberrant expression of miRNAs is associated with many human diseases such as cancer, Down syndrome, Alzheimer's disease, autoimmune diseases, to name a few. An example of miRNAs upregulated in hematological malignancies are shown in Table 1 below. Anti-miRNA PNA oligomers can be designed based on a portion or full sequence of any known targeted miRNA as described herein to form a triplex PNA/RNA/PNA, inhibiting expression of the targeted miRNA.

TABLE 1
MIRNAS UPREGULATED IN HEMATOLOGICAL MALIGNANCIES

| | | | SEQ ID |
|---|---|---|---|
| Name | Cancer | Sequence | NO: |
| miR-21 | CLL, CML, | UAGCUUAUCAGACUGAUGUUGA | 8 |
| | lymphoma, | | |
| | Glioblastoma | | |
| miR-221 | CLL | ACCUGGCAUACAAUGUAGAUUU | 9 |
| miR-888 | Endometrial, breast, | UACUCAAAAAGCUGUCAGUCA | 10 |
| | prostate | | |
| miR-155 | CLL, B-cell | UUAAUGCUAAUCGUGAUAGGGGUU | 11 |
| | lymphoma | | |
| miR-10b | Glioblastoma, MDS | UACCCUGUAGAACCGAAUUUGUG | 12 |
| miR-147b | Lung cancer | UGGAAACAUUUCUGCACAAACU | 13 |
| miR-122 | CTCL | UGGAGUGUGACAAUGGUGUUUG | 14 |
| miR-22 | AML | AGUUCUUCAGUGGCAAGCUUUA | 15 |
| miR-99 | AML | AACCCGUAGAUCCGAUCUUGUG | 16 |
| miR-128 | Leukemia | CGGGGCCGUAGCACUGUCUGAGA | 17 |
| miR-182 | T-ALL | UUUGGCAAUGGUAGAACUCACACU | 18 |
| miR-221 | CLL | ACCUGGCAUACAAUGUAGAUUU | 19 |
| miR-222 | CLL | CUCAGUAGCCAGUGUAGAUCCU | 20 |
| miR-4262 | AML | CUAGGAGGCCUUGGCC | 21 |
| miR-20 | AML | UAAAGUGCUUAUAGUGCAGGUAG | 22 |
| miR-125 | MM, CTCL | UCCCUGAGACCCUAACUUGUGA | 23 |
| miR-17 | ALL, CML | CAAAGUGCUUACAGUGCAGGUAG | 24 |
| miR-142 | MM | CAUAAAGUAGAAAGCACUACU | 25 |
| miR-181a | Lymphoma | AACAUUCAACGCUGUCGGUGAGU | 26 |
| miR-187 | Lymphoma | GGCUACAACACAGGACCCGGGC | 27 |
| miR-30 | Classical Hodgkin | UGUAAACAUCCUCGACUGGAAG | 28 |
| | lymphoma | | |

By “expression” or “gene expression,” it is meant the overall flow of information from a gene (without limitation, a functional genetic unit for producing a gene product, such as RNA or a protein in a cell, or other expression system encoded on a nucleic acid and comprising: a response elements and/or enhancers; an expressed sequence that typically encodes a protein (open-reading frame or ORF) or functional/structural RNA, and a polyadenylation sequence), to produce a gene product (typically a protein, optionally post-translationally modified or a functional/structural RNA). The designated sequence may be all or part of the RNA and may wholly or partially regulate and/or affect the translation or transcription of a gene.

Use of the PNA oligomers to target an RNA in a cell, such as an mRNA or miRNA, will inhibit expression or the RNA at the translational stage in the case of mRNA, and/or affect gene expression by downregulation or upregulating expression of the miRNA and its downstream effects on its target genes.

As shown herein below, reduction in miR-155 expression in multiple lymphoma cell lines in vitro and in xerograft mouse models in vivo produced by uptake of a gPNA with a tail-clamp capable of forming a triplex PNA/RNA/PNA with miR-155 in multiple lymphoma cell lines, resulted in a decrease in tumor cell proliferation and viability that is dose-dependent, showing the superior binding affinity of the anti-miR-155-PNA described herein, resulting in a strong anti-miR-155 effect.

Therefore, in an aspect, a method for increasing or decreasing expression of miRNA-155 target genes, or associated oncogenes. In one aspect, a method for increasing gene expression of an miR-155 associated tumor suppressor gene comprising downregulating miR-155 expression by providing to a cell a PNA oligomer described herein. In an aspect, the tumor suppressor gene is any of CSF1R, CUX1, and SHIP1.

In another aspect, a method for reducing expression of miR-155 associated oncogenes comprising downregulating miR-155 expression in a cell comprising providing to the cell a PNA oligomer as described herein, wherein the miR-155 associated oncogene is MCL1.

In yet another aspect, a method for increasing gene expression of miR-155 associated oncogenes comprising downregulating miR-155 expression by providing to a cell a PNA oligomer described herein. In an aspect, the miR-155 associated oncogene is Caspase-3.

In another aspect, a method for increasing apoptosis of tumor cells overexpressing miR-155, comprising downregulating miR-155 expression by providing to a cell a PNA oligomer of described herein.

The disclosed compositions can be used for ex vivo or in vivo. The methods typically include contacting a cell with an effective amount of PNA oligomers, optionally in combination with a potentiating agent, to modify the expression of an RNA. As discussed in more detail below, the contacting can occur ex vivo or in vivo. In preferred embodiments, the method includes contacting a population of target cells with an effective amount of the composition, to modify the expression of RNA to achieve a therapeutic result.

For example, the effective amount or therapeutically effective amount can be a dosage sufficient to treat, inhibit, or alleviate one or more symptoms of a disease or disorder, or to otherwise provide a desired pharmacologic and/or physiologic effect, for example, reducing, inhibiting, or reversing one or more of the underlying pathophysiological mechanisms underlying a disease or disorder. The molecules can be administered in an effective amount to induce formation of a PNA/RNA/PNA triplex at the target site.

The formulation of the composition comprising the PNA oligomers is made to suit the mode of administration.

Pharmaceutically acceptable carriers are determined in part by the particular composition being administered, as well as by the particular method used to administer the composition. Accordingly, there is a wide variety of suitable formulations of pharmaceutical compositions containing the nucleic acids. The precise dosage will vary according to a variety of factors such as subject-dependent variables (e.g., age, immune system health, clinical symptoms etc.).

The disclosed compositions can be administered to or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times about week.

In general, by way of example only, dosage forms useful in the disclosed methods can include doses in the range of about 10² to about 10⁵⁰, or about 10⁵ to about 10⁴⁰, or about 10¹⁰ to about 10³⁰, or about 10¹² to about 10²⁰ copies of triplex-forming molecules per dose.

The disclosed compositions can be administered directly to a subject for in vivo gene therapy.

The disclosed compositions are preferably employed for therapeutic uses in combination with a suitable pharmaceutical carrier. Such compositions include an effective amount of the composition, and a pharmaceutically acceptable carrier or excipient.

The disclosed compositions of PNA oligomers may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. The particles can be capable of controlled release of the active agent. The particles can be microparticle(s) and/or nanoparticle(s). The particles can include one or more polymers. One or more of the polymers can be a synthetic polymer. The particle or particles can be formed by, for example, single emulsion technique or double emulsion technique or nanoprecipitation. Such systems are well known to those skilled in the art and may be optimized for use with the appropriate nucleic acid.

Various methods for nucleic acid or PNA delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994).

Targeting molecules can be proteins, peptides, nucleic acid molecules, saccharides or polysaccharides that bind to a receptor or other molecule on the surface of a targeted cell. The degree of specificity and the avidity of binding to the target cells can be modulated through the selection of the targeting molecule. For example, antibodies are very specific. These can be polyclonal, monoclonal, fragments, recombinant, or single chain, many of which are commercially available or readily obtained using standard techniques.

Examples of moieties include, for example, targeting moieties which provide for the delivery of molecules to specific cells, e.g., antibodies to hematopoietic stem cells, CD34+ cells, epithelial cells, T cells or any other preferred cell type, as well as receptor and ligands expressed on the preferred cell type. In some embodiments, the moieties target hematopoietic stem cells. The choice of targeting molecule will depend on the method of administration of the particle composition and the cells or tissues to be targeted. The targeting molecule may generally increase the binding affinity of the particles for cell or tissues or may target the particle to a particular tissue in an organ or a particular cell type in a tissue.

The PNA delivery system can be provided to the cell either directly, such as by contacting it with the cell, or indirectly, such as through the action of any biological process. The PNA delivery system can be provided to the cell by endocytosis, receptor targeting, coupling with native or synthetic cell membrane fragments, physical means such as electroporation, combining the PNA delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the PNA delivery system can be provided to the cell using techniques such as antibody-related targeting and antibody-mediated immobilization of a viral vector.

Formulations for topical administration may include ointments, lotions, creams, gels, drops, suppositories, sprays, liquids and powders. Conventional pharmaceutical carriers, aqueous, powder or oily bases, or thickeners can be used as desired.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions, solutions or emulsions that can include suspending agents, solubilizers, thickening agents, dispersing agents, stabilizers, and preservatives. Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butanediol, Ringer's dextrose, dextrose and sodium chloride, lactated Ringer's or fixed oils. Intravenous vehicles include fluid and nutrient replenishers, and electrolyte replenishers (such as those based on Ringer's dextrose). Preservatives and other additives may also be present such as, for example, antimicrobials, antioxidants, chelating agents and inert gases. In addition, sterile, fixed oils are conventionally employed as a solvent or suspending medium. For this purpose, any bland fixed oil including synthetic mono- or di-glycerides may be employed. In addition, fatty acids such as oleic acid may be used in the preparation of injectables. Carrier formulation can be found in Remington's Pharmaceutical Sciences, Mack Publishing Co. Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. In one embodiment, the triplex-forming molecules and/or donor oligonucleotides are conjugated to lipophilic groups like cholesterol and lauric and lithocholic acid derivatives with C32 functionality to improve cellular uptake. For example, cholesterol has been demonstrated to enhance uptake and serum stability of siRNA in vitro and in vivo. In addition, it has been shown that binding of steroid conjugated oligonucleotides to different lipoproteins in the bloodstream, such as LDL, protect integrity and facilitate biodistribution. Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fc (II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

In general, methods of administering compounds, including oligonucleotides and related molecules, are well known in the art. In particular, the routes of administration already in use for nucleic acid therapeutics, along with formulations in current use, provide preferred routes of administration and formulation for the PNA oligomers described above.

The disclosed compositions can be administered by a number of routes including, but not limited to, oral, intravenous, intraperitoneal, intramuscular, transdermal, subcutaneous, topical, sublingual, rectal, intranasal, pulmonary, and other suitable means. The compositions can also be administered via liposomes. Such administration routes and appropriate formulations are generally known to those of skill in the art.

Administration of the formulations may be accomplished by any acceptable method which allows the PNA oligomer compositions to reach their targets.

Any acceptable method known to one of ordinary skill in the art may be used to administer a formulation to the subject. The administration may be localized (i.e., to a particular region, physiological system, tissue, organ, or cell type) or systemic, depending on the condition being treated.

Injections can be e.g., intravenous, intradermal, subcutaneous, intramuscular, or intraperitoneal. In some embodiments, the injections can be given at multiple locations. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, cither alone or attached to a carrier that can be absorbed. For systemic administration, it may be preferred that the composition is encapsulated in liposomes.

The compositions may be delivered in a manner which enables tissue-specific uptake of the agent and/or nucleotide delivery system.

Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

The formulations may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations. Administration of the formulations using such a delivery system may be, for example, by oral dosage forms, bolus injections, transdermal patches or subcutaneous implants. Maintaining a substantially constant concentration of the composition may be preferred in some cases.

Other delivery systems suitable include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, poly anhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include erosional systems in which the oligonucleotides are contained in a formulation within a matrix (for example, as described in U.S. Pat. Nos. 4,452,775, 4,675,189, 5,736, 152, 4,667,013, 4,748,034 and 5,239,660), or diffusional systems in which an active component controls the release rate (for example, as described in U.S. Pat. Nos. 3,832,253, 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the triplex-forming molecules and donor oligonucleotides. In addition, a pump-based hardware delivery system may be used to deliver one or more embodiments.

Suitable subjects include, but are not limited to, mammals such as a human or other primate, a rodent such as a mouse or rat, or an agricultural or domesticated animal such as a dog, cat, cow, horse, pig, or sheep. The subject can be an adult, child, infant, or a multi-cell or single-cell embryo. The methods can include in utero delivery of the composition to an embryo or fetus in need thereof.

The invention is further illustrated by the following non-limiting examples.

### EXAMPLES

### Methods

Synthesis of PNA oligomers: Boc-protected regular monomers (for PNA-155) and serine gamma monomers used for gamma tcPNA-155, gamma PNA-155 and Scr-γtcPNA-155 synthesis were purchased from ASM Research Chemicals GmbH (Hannover, Germany). The monomers were vacuum dried prior to start of solid-phase synthesis. Around 100 mg arginine-loaded resin was soaked in dichloromethane (DCM) for 5 hours in a reaction vessel. The DCM was drained, and the resin was deprotected using trifluoroacetic acid-m-cresol (95:5) mixture for 5 mins. This deprotection step was repeated two additional times followed by washing the resin with DCM and N,N-dimethylformamide (DMF). The monomer was dissolved in a coupling solution comprising of a mixture of 0.2M N-methyl pyrrolidone (NMP), 0.52M Di-isopropylethylamine (DIEA), and 0.39M O-benzotriazole-N,N,N′,N′-tetramethyl-uronium-hexafluoro-phosphate (HBTU). The coupling solution was added to the reaction vessel and rocked for 2 hrs. The resin was capped using a capping solution (a mixture of NMP, Pyridine, and acetic anhydride). The resin was washed with DCM (8×). The entire process was repeated until the last monomer was added. 5-carboxytetramethylrhodamine (TAMRA) was conjugated to N terminus of gamma tcPNA-155. The PNA was cleaved from the resin using a cleavage cocktail (thioanisole, m-cresol, TMFSA, TFA (1:1:2:6), and the vessel was rocked for 1.5 hrs. The PNA was collected and precipitated using diethyl ether and centrifuged at 3500 rpm for 5 mins. The PNA was washed with ether twice and vacuum dried. The PNA was purified by RP-HPLC and absorbance of the PNA was measured using Nanodrop™ One (Thermofisher Scientific, MA). The extinction coefficient of the individual monomers used for calculating PNA concentration (6,600 M⁻¹ cm⁻¹ (C), 13,700 M⁻¹ cm⁻¹ (A), 8,600 M⁻¹ cm⁻¹ (T), 11,700 M⁻¹ cm⁻¹ (G)).

Gel shift assay: Different concentration of PNA-155 or gamma tcPNA-155 were made in physiological buffer (2 mM MgCl₂, 150 mM KCl, 10 mM NaPi) and incubated overnight at 37° C. with 1 μM DNA-155 target. The samples were run through 8% Polyacrylamide gel electrophoresis (PAGE) gel in IX TBE buffer at 120 volts for 35 mins. The gel was stained with SyBr Gold for two mins and visualized using Gel Doc™ EZ Imager (Bio-rad). The PNA-entrapped NDs were prepared at PNA: lipid molar ratios of 1:200, 1:500, 1:1000 and 1:2500, homogeneously dispersed in chloroform. After being dried, the sample was re-hydrated in water to form a 10 wt. % mother solution by a proper temperature cycling and vortexing. The solution was then centrifuged at 10,000 rpm for 10 minutes in a Beckman Counter centrifuge to separate the aggregates and large particles and diluted to the desired concentration prior to the use for studies.

Cellular Uptake: U2932 are suspended cell lines and were purchased from Leibniz Institute (DSMZ, Germany). The cells were regularly tested for Mycoplasma contamination using MycoAlert Mycoplasma detection kit (Lonza). The authenticity of the cell lines was confirmed by Human cell STR profiling service by ATCC. All the cells used in the experiment were passaged less than 8 times. 50,000 U2932 cells were seeded in twenty four well plate (37° C. and 5% CO₂). The cells were treated with gamma tcPNA-155 TAMRA (500 nM concentration). After 48 hrs, the cells were washed twice with PBS and then fixed using 4% paraformaldehyde (PFA) for 10 mins at room temperature. The cells were washed with PBS and then permeabilized using 0.1% Triton™ X for 10 mins at room temperature. The cells were washed with PBS and the cells were finally resuspended in 50 mL PBS. A drop of mounting media with DAPI (Life technologies) was placed on the slide. 10 mL of cells were mixed with the drop of DAPI on the slide and coverslip was placed on the slide. The slide was allowed to dry overnight and imaged using Keyence digital microscope.

For evaluating cellular uptake by flow cytometry, 400,000 U2932 cells were collected in a 12 well plate (37° C., 5% CO2). Three wells were untreated and three other cells were treated with 500 nM gamma tcPNA-155 TAMRA. After 48 hrs, the cells were washed twice with PBS and then fixed using 4% paraformaldehyde (PFA) for 10 mins at room temperature. The cells were passed through the FACS tube. Further, analysis was performed using LSR Fortessa™ X-20 Cell analyzer (BD Bioscience). The FACS data was plotted using FlowJo software.

Gene expression by RT-PCR: 400,000 U2932 cells were seeded in a 12 well plate. The cells were treated with 500 nM PNA-155, gamma tcPNA-155, gamma PNA-155, Scr gamma tcPNA-155 or were PBS treated (control) for 48 hrs in an incubator (37° C. and 5% CO2). The cells were centrifuged at 2000 rpm for 4 mins at 4° C. The total RNA from the cell pellet was extracted using RNeasy® mini kit (Qiagen). The cDNA was prepared in thermal cycler (Bio-rad) using reverse transcriptase, RNase inhibitor, dNTPs, nuclease-free water and RT primers specific for miR-155 and U6. Random primers were used for the preparation of cDNA for downstream targets. The cDNA was amplified using miR-155 assay, U6 assay, or specific downstream target assays in CFX Connect™ Real-time PCR detection system (Bio-rad). The samples were subjected to polymerase activation (95° C. for 10 mins), followed by 40 cycles of denaturation (95° C. for 15 sec) and annealing (60° C. for 1 min). The 2^{-ΔΔCT} method was used to calculate the fold change in target genes.

Similarly, 400,000 SUDHL-2 cells were seeded in a 12 well plate and treated with 500 nM Scr gamma tcPNA-155, PNA-155 and gamma tcPNA-155 for 48 hrs in an incubator (37° C. and 5% CO₂). The RNA was extracted and the samples for gene expression were prepared in the same manner as described above.

Cell viability by trypan blue assay: Diffused large B cell lymphoma (DLBCL) cell lines like SUDHL-2 (ATCC® CRL-2956™) and SUDHL-5 (ATCC® CRL-2958™) cells were purchased from ATCC (Virginia, USA). The cells were regularly tested for Mycoplasma contamination using MycoAlert™ Mycoplasma detection kit (Lonza). All the cells used in the experiment were passaged less than 8 times. 10,000 U2932, SUDHL-5, or SUDHL-2 cells were plated in a 96 well plate. The cells were treated with different doses (500 nM, 1000 nM, 2000 nM and 4000 nM) of PNA-155, gamma tcPNA-155 or Scr-γtcPNA-155 for 48 hrs in an incubator (37° C. and 5% CO₂). The dead cells were marked with trypan blue. Further counting was performed using an automated cell counter (Bio-rad).

Apoptosis Assay: 400,000 U2932 cells were seeded in a 12 well plate. The cells were treated with 500 nM PNA-155, γPNA-155, γtcPNA-155, Scr-γ tcPNA-155 or were PBS treated (control) for 48 hrs in an incubator (37° C., 5% CO₂). The cells were washed with PBS twice. The cells were centrifuged at 2000 rpm for 4 mins at 4° C. The cell pellet was suspended in IX Annexin V binding buffer. The cells were then counted and 100 μL of cell suspension (containing 2.5×10⁵ cells) was passed through the FACS tube. The cells were stained with 12.5 uL Phycoerythrin (PE) Annexin dye and 12.5 μL 7-Amino-Actinomycin (7AAD) and kept in dark for 15 mins. 400 μL of IX Annexin V binding buffer was added to the cells and the cells were then analyzed using LSR Fortessa™ X-20 Cell analyzer as indicated above.

For Annexin V FITC stained fluorescent imaging method: 10,000 U2932 cells were seeded in 96 well plate (37° C. and 5% CO₂). The cells were treated with Scr-γtcPNA-155, PNA-155, γPNA-155, γtcPNA-155 (500 nM concentration) for 48 hours. Annexin V FITC diluted 1:10 in 1× Annexin binding buffer was supplemented to each well. The plate was kept at room temperature for 15 mins. The cells were imaged using 10× lens on Keyence digital microscope.

### Western Blot

400,000 U2932 cells were collected in a 12 well plate and treated with 500 nM PNA-155, γPNA-155, γtcPNA-155 or Scr-γtcPNA-155 for 48 hrs in an incubator. The cell pellet was collected by centrifuging at 2000 rpm for 4 mins at 4° C. 1×RIPA buffer and 1× protease inhibitor were added to the cell pellet and subjected to intermittent vortexing after 10 mins (3×) to extract the proteins from the cell pellet. The protein was collected after centrifuging the tube at 10,000 rpm for 10 mins at 4° C. The protein concentration was measured by Lowry protein assay. About 25 μg protein was loaded on SDS PAGE gel (4-20% MP TGX stain-free gels, Bio-rad) and separated at 101 Volts for 90 mins. The proteins were transferred from the gel to a PVDF membrane at 110 Volts for 90 mins. The PVDF membrane was blocked using 5% milk in 1×TRIS buffered saline for 1 hr. The Western blotting was performed using the following antibodies: Mcl-1 (39224), CASP3 (9622S), Vinculin (13901), antirabbit IgG HRP linked antibody (7074) (Cell Signaling Technology). The blots were imaged using ChemiDoc™ Imaging System (Bio-rad). Protein expression intensities were determined using Image J software and normalized relative to loading control and treatment control.

Safety assessment by trypan blue assay Primary blood mononuclear cells (PBMC) (ATCC® PCS-800-011™) were purchased from ATCC (Virginia, USA). The cells were regularly tested for Mycoplasma contamination using MycoAlert™ Mycoplasma detection kit (Lonza). All the cells used in the experiment were passaged less than 2 times. 10,000 PBMC cells were seeded in a 96 well plate. The cells were treated with 500 nM, 1000 nM, 2000 nM and 4000 nM PNA-155 and γtcPNA-155 for 48 hrs in an incubator. The dead cells were examined with trypan blue and further counted using an automated cell counter (Bio-rad).

Study approval: All the animals' experiments were performed at the University of Connecticut, Storrs campus, in compliance and approved by the Institutional Animal Care and Use Committee (IACUC). The authorization number for approved IACUC protocol is A21-041.

Mouse tumor xenograft: Female NSG mice (NOD.Cg-Prkdcscid Il2rgtm1Wjl/SzJ, strain 005557) weighing 20-23 g were procured from Jackson laboratories. 1×10⁷ U2932 cells suspended in RPMI-1640 medium were injected subcutaneously in the right and left flank of 6-week-old NSG mice. The mice developed a small bump in 2 weeks. When the tumor volume reached 100-200 mm³, the mice were randomly assigned to the treatment group (n=6 per group).

For SUDHL-2 xenografts, 1×10⁷ SUDHL-2 cells suspended in RPMI-1640 medium were injected subcutaneously in the right and left flank of 6-week-old NSG mice. The mice developed a small bump in 2-3 weeks. When the tumor volume reached 100-200 mm³, the mice were randomly allocated to the treatment group (n=5 per group).

Biodistribution: The γtcPNA-155 TAMRA PNA was intra-tumorally injected in the NSG mice (n=3) bearing 200 mm³ tumors at the dose of 3 mg kg-1 dose at an interval of 1 week. When the tumor volume reached 2000 mm³, the mice were euthanized by CO₂ inhalation. The tumors were harvested and imaged using IVIS Spectrum system. The tumors were embedded in OCT compound. The tumors were cryosectioned at 10 mm thickness using Leica cryostat. The tumor sections were fixed using 4% formaldehyde for 10 mins, followed by washing with PBS for 10 mins. The tumor sections were then permeabilized using 0.2% Triton™ X for 20 mins followed by washing with PBS. A drop of mounting media with DAPI (Life technologies) was placed on the tumor section and a coverslip was placed on it. The tumor sections were imaged using Keyence digital microscope.

RNA and Protein extraction from tumor samples: The mice were injected intra-tumorally with 1 mg kg-1 dose of PNA-155, γtcPNA-155, γPNA-155, Scr-γtcPNA-155 or were untreated. The injections were repeated two additional times after 1 week each. The length, breadth, and depth of the tumors were measured daily using a vernier caliper. The mice were euthanized when the tumor volumes reached 2000 mm³. The resected tumor sections were finely minced using a sterile blade and suspended in dissociation media (4 ml) comprising of RPMI-1640, 1.2 mg/ml dispase, and 0.5 mg/ml collagenase for 90 mins at 500 rpm at 37° C. The dissociated tumors were washed with buffer saline at 2500 rpm (4 mins) at 4° C. and then suspended in 0.25% trypsin for 4 mins at room temperature. RPMI 1640 media was added to the trypsinized tumor mass, and the cells were passed through a 70 um filter. The cells were centrifuged at 2500 rpm for 4 mins at 4° C. The cell pellet was then suspended in 1×RBC lysis buffer (Sigma) and incubated on ice for 10 mins. PBS was added to the cells and the cells were passed through a 40 um filter. The cells were centrifuged at 2500 rpm for 4 mins at 4° C. The cell pellet was resuspended in 0.5% BSA in PBS. The mouse cells were removed from the tumor cells using a mouse cell depletion kit (Miltenyi Biotec) according to the manufacturer's protocol. The enriched tumor cells from each tumor sample were divided into two fractions. RNA for gene expression analysis was extracted from one tumor fraction by the same procedure as mentioned earlier. The protein for western blot analysis was extracted from the second fraction using the method described above.

Histopathology and immunohistochemistry; The mice were sacrificed by CO₂ inhalation when tumor volume reached 2000 mm³. The tumor and vital organs (e.g., liver, kidney, spleen, lungs, heart) were carefully isolated, weighed, and fixed in the 10% NBF solution. The sections (5 μM) of formalin-fixed paraffin-embedded liver and kidney were stained by hematoxylin and eosin for the histological analysis. The sections of 5 μM of the formalin-fixed paraffin-embedded tumor were heated (95° C., 20 min) in citrate buffer (10 mM) for antigen recovery. Further incubation was performed with primary antibodies. The concentrations of rabbit anti-Ki-67 (D2H10) and rabbit anti-Caspase-3 (9962) were 1:100. The antigen-primary antibody complexes were examined by fluorescent tagged secondary antibodies. Images were taken using a Zeiss confocal microscope (LSM 510).

Statistical analysis: Graphpad Prism 9 software (Version 9.2) was used for all statistical analyses. The data are reported as means SEM of triplicates, and the numbers and replicates are included in the Figure captions. Unpaired two-tailed and multiple student t-test was performed for experiments. For in vivo studies, animals were randomly assigned in groups to minimize the bias, and based on prior experience with animal models, sample size was selected. The statistical comparisons are significant when *p<0.05, **p<0.01, ***p<0.001, ****p<0.0001.

### Example 1: Design and Synthesis of Anti-Mir-155 Gamma tcPNA

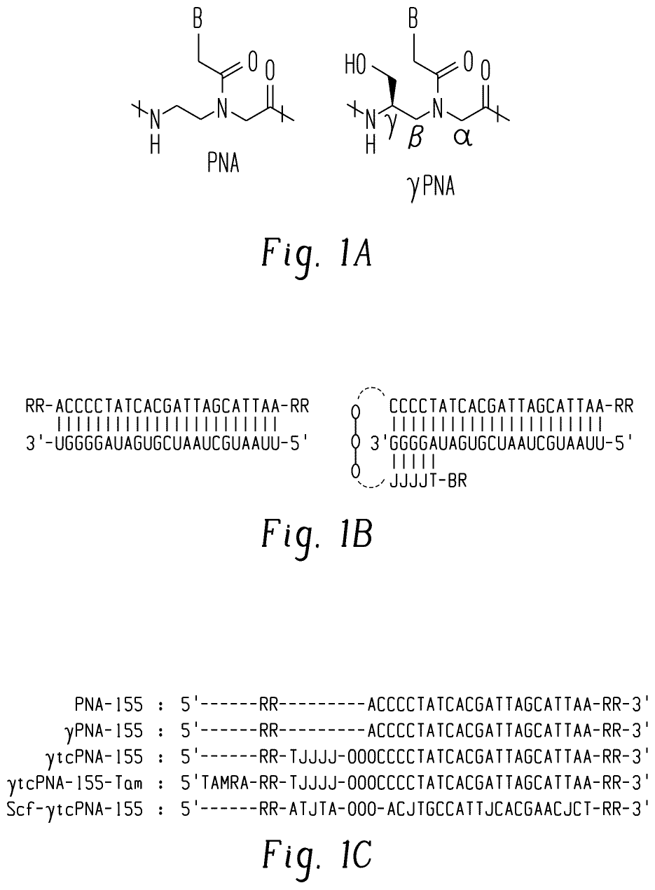

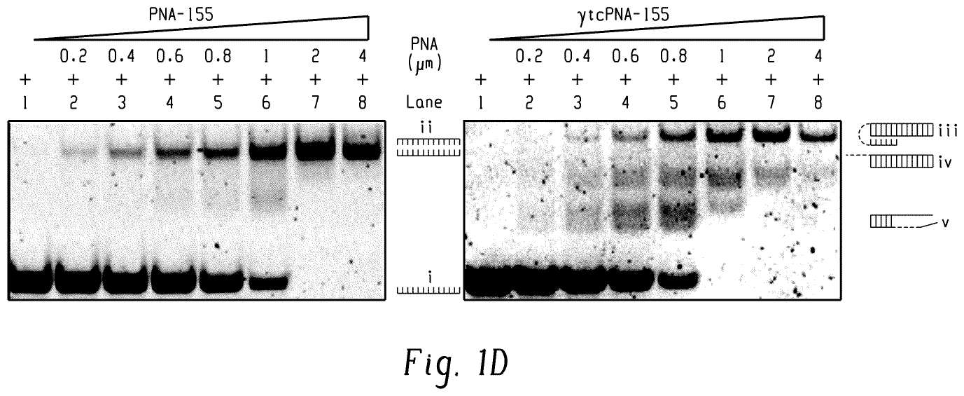

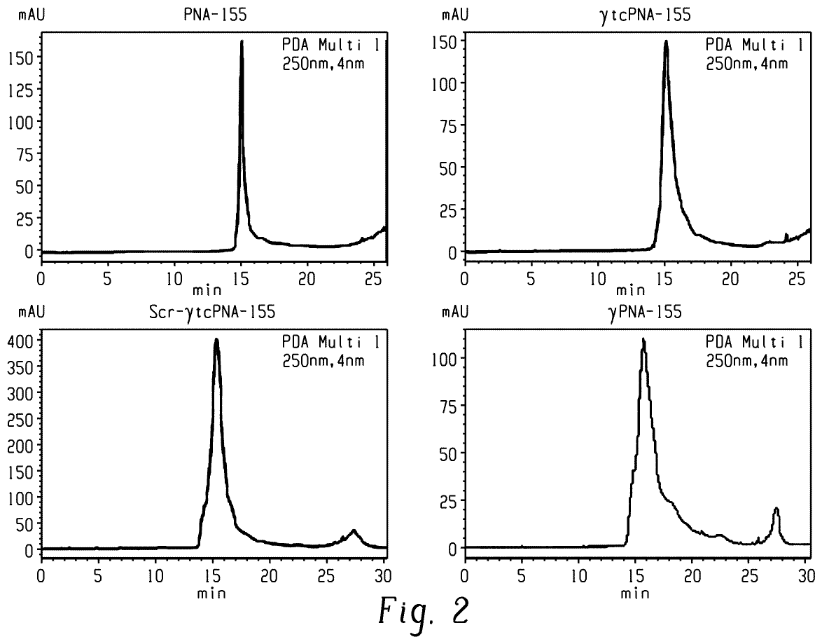

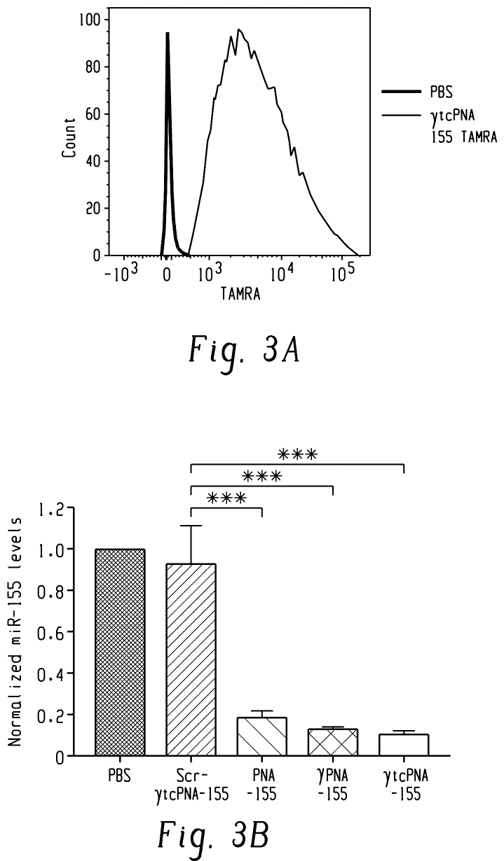

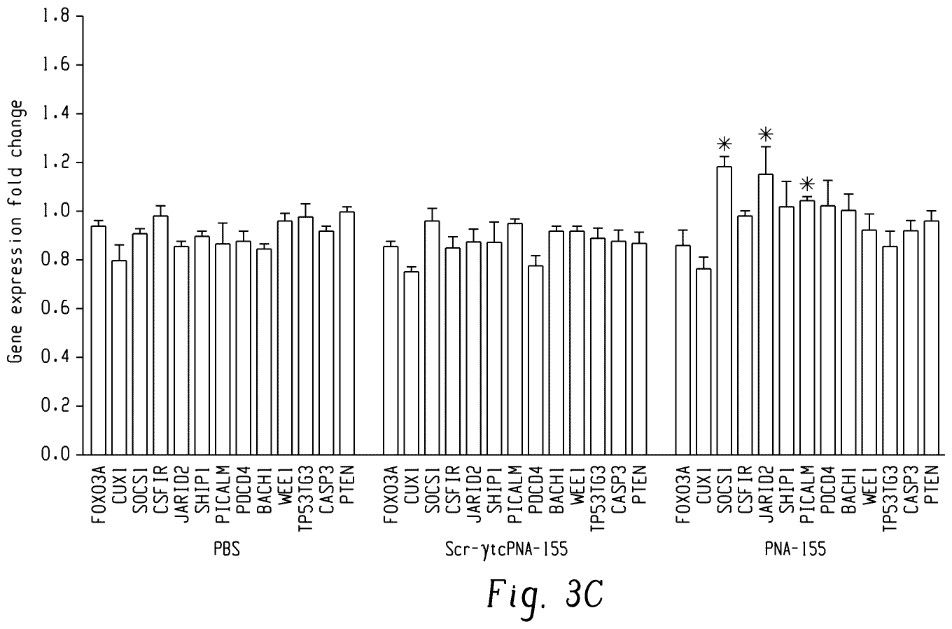

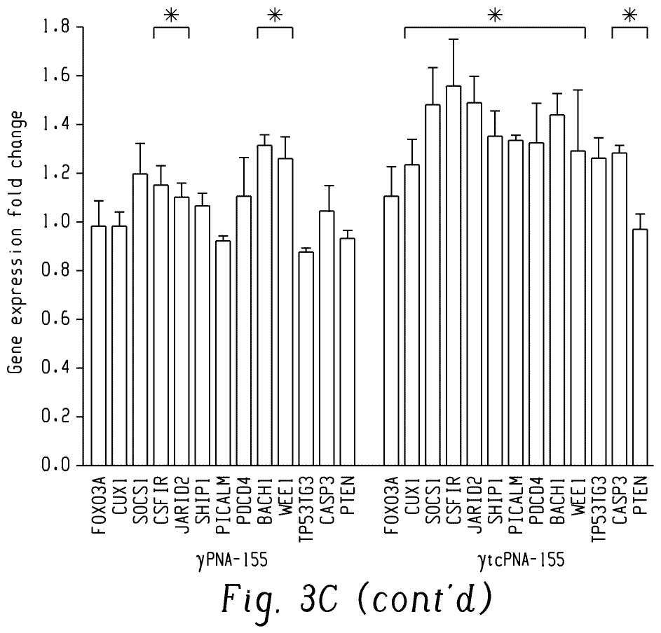

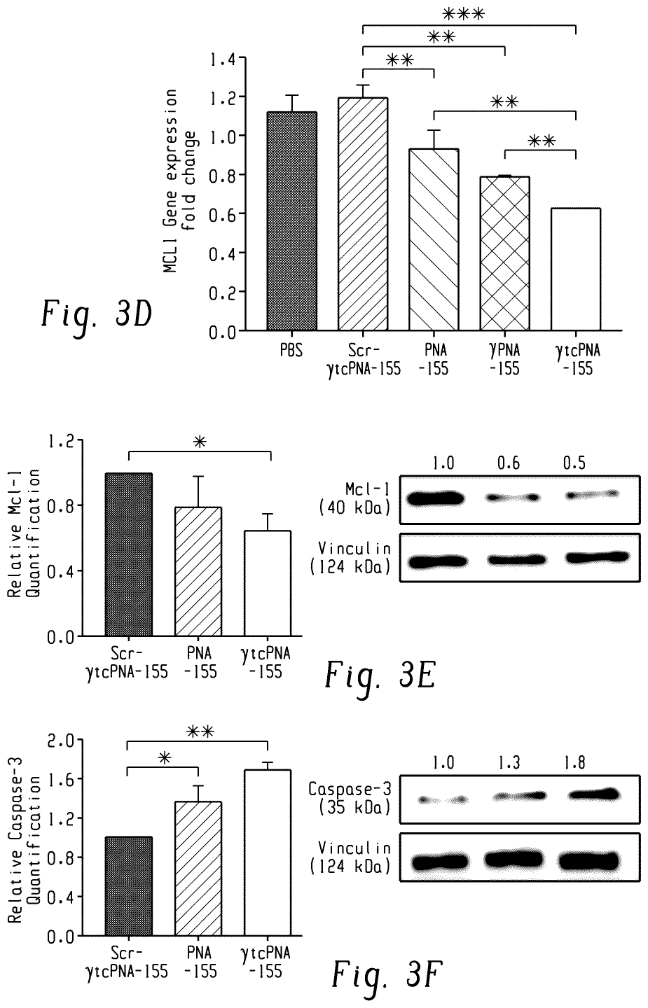

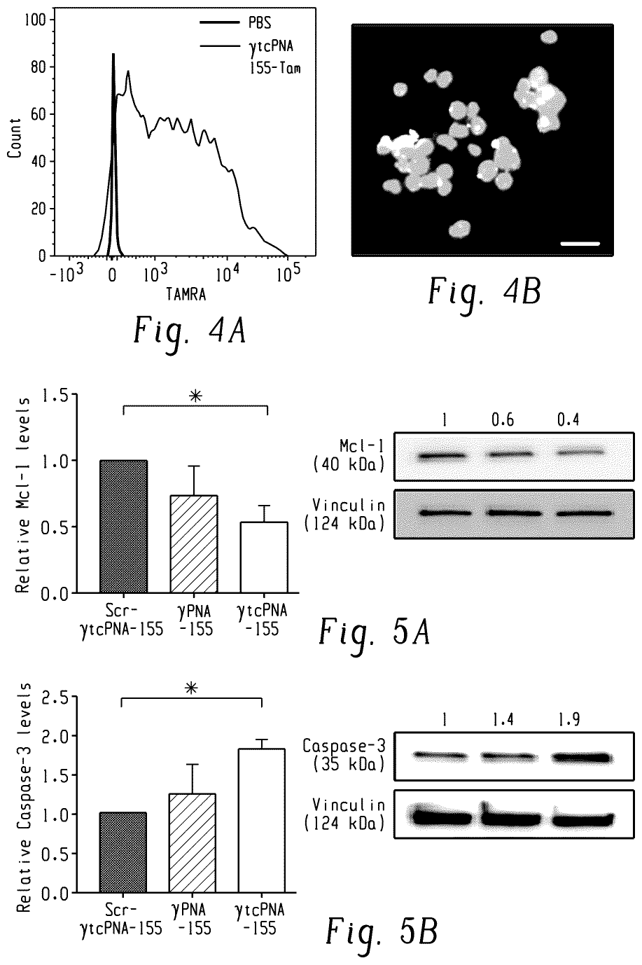

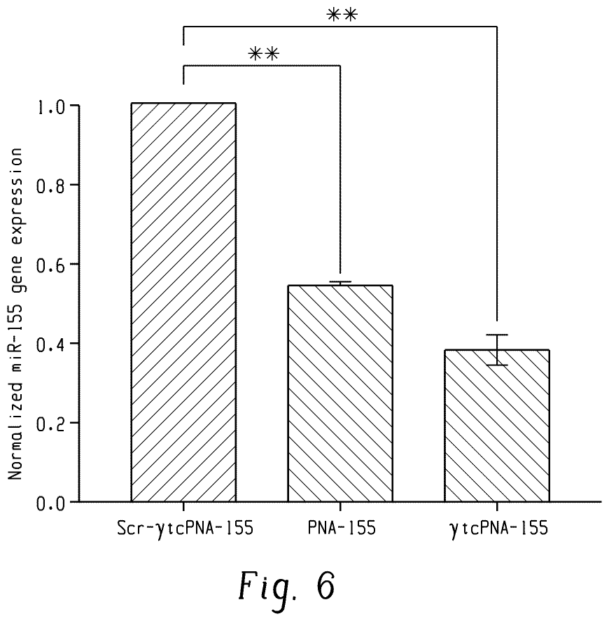

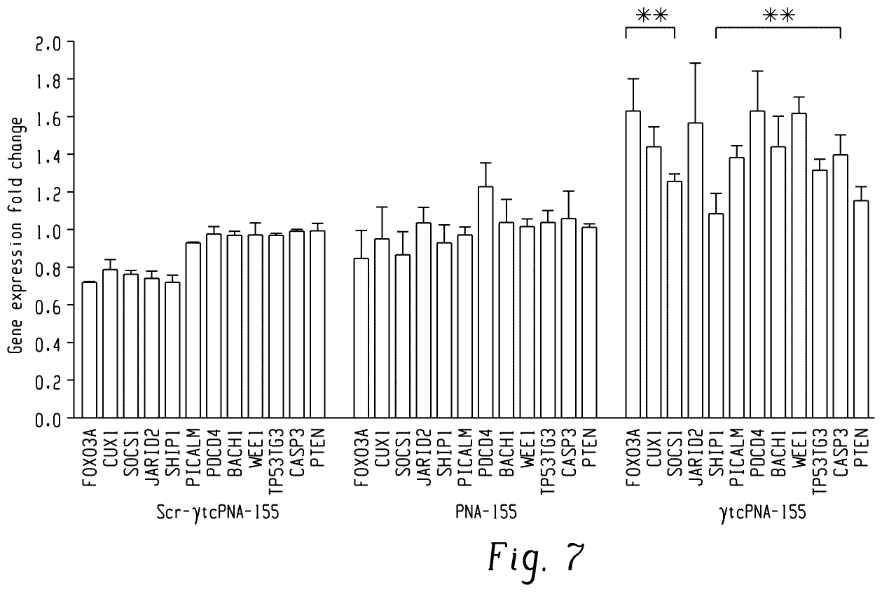

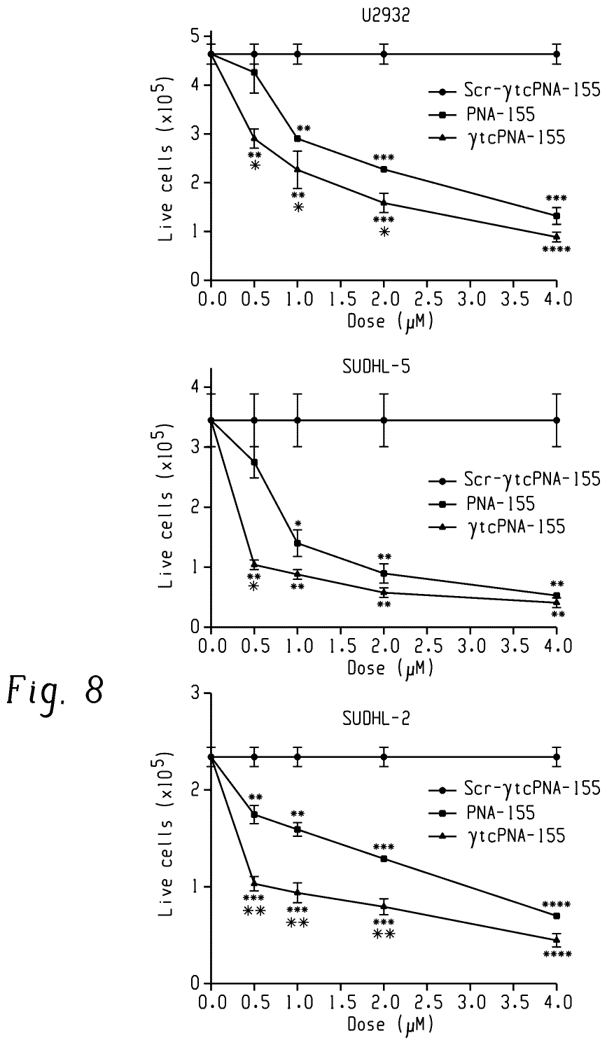

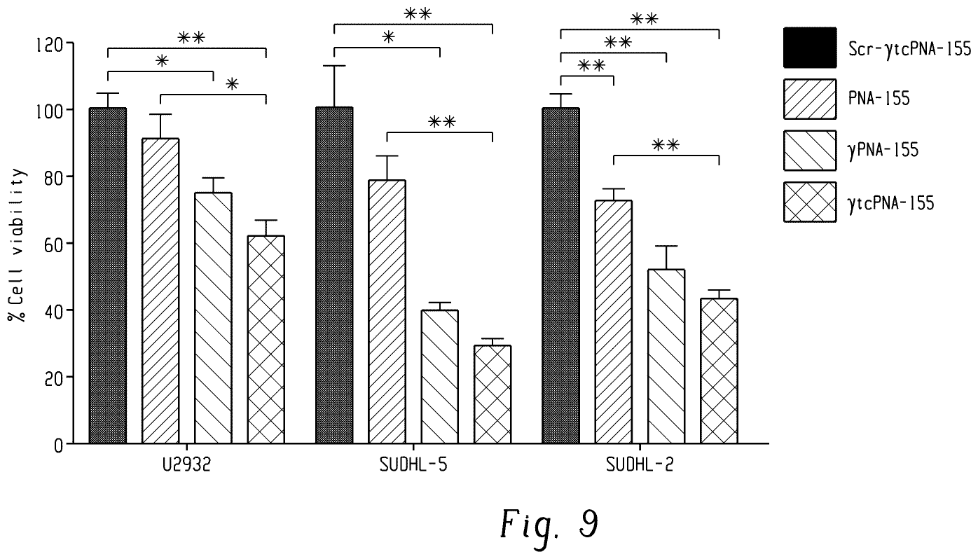

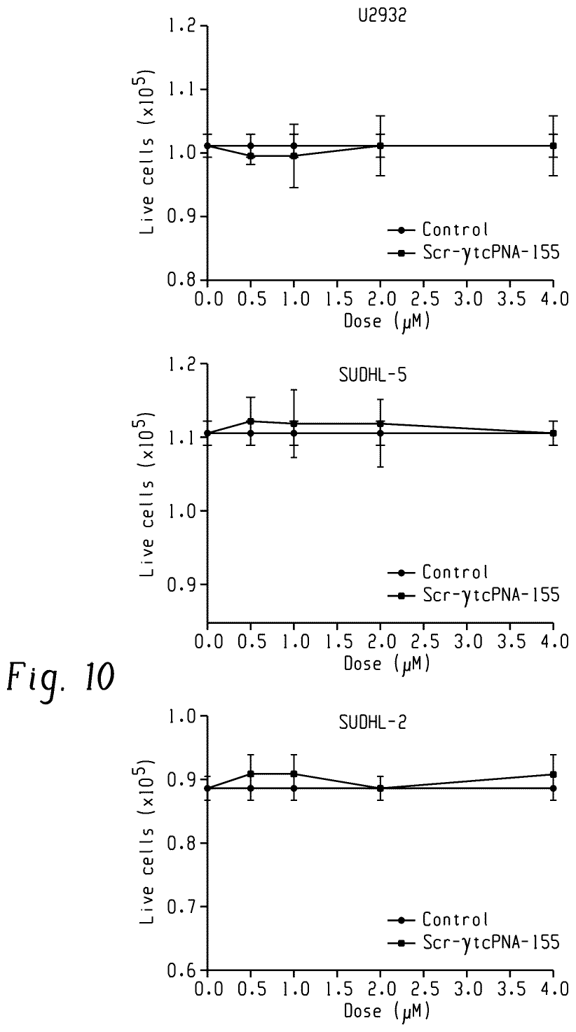

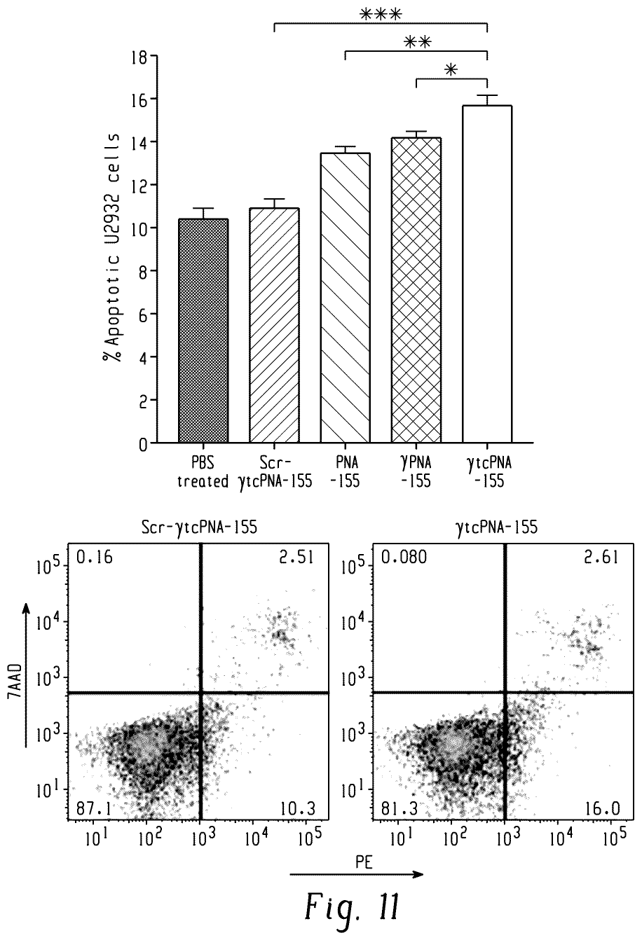

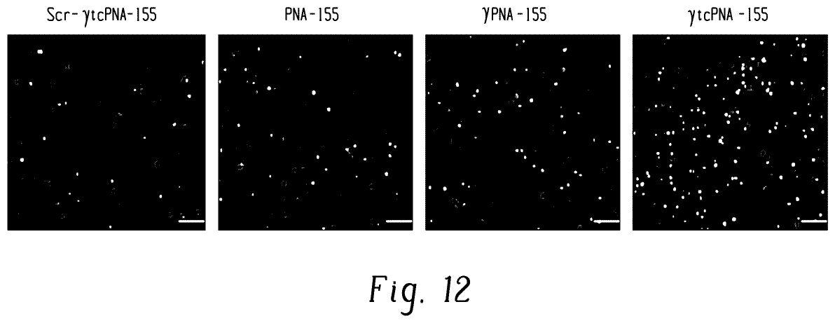

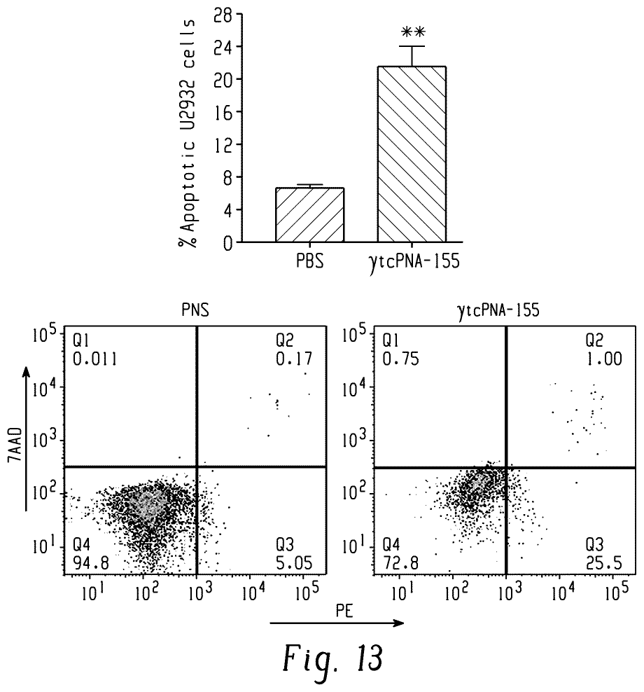

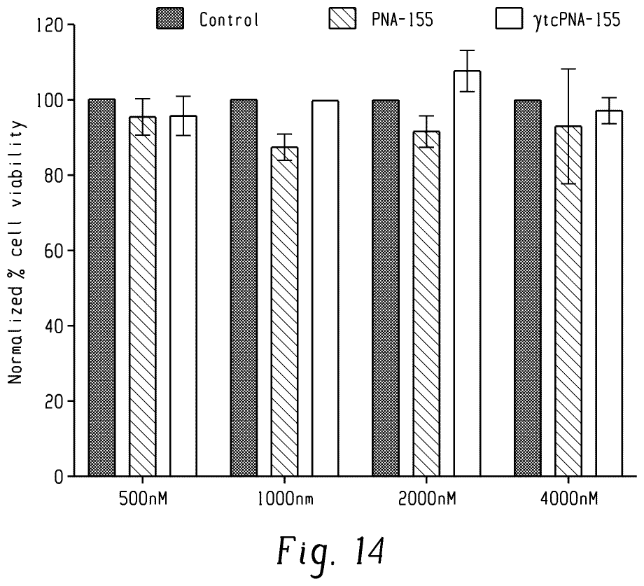

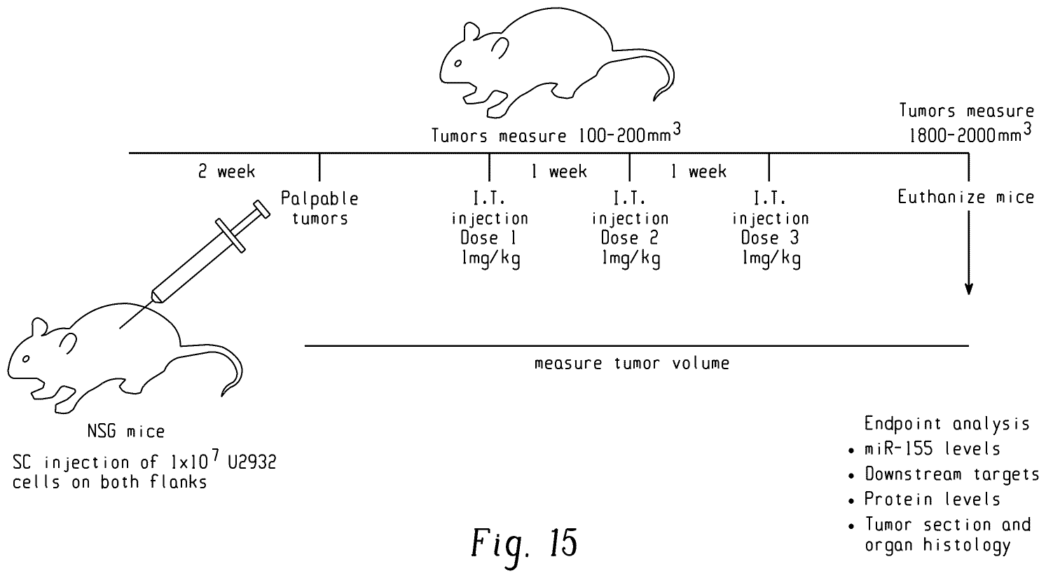

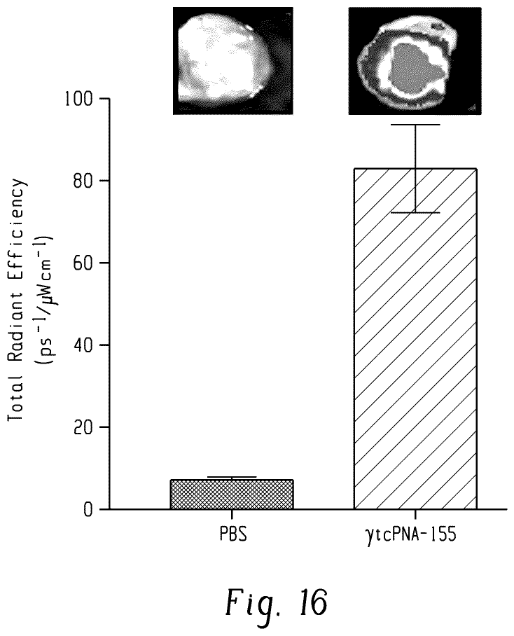

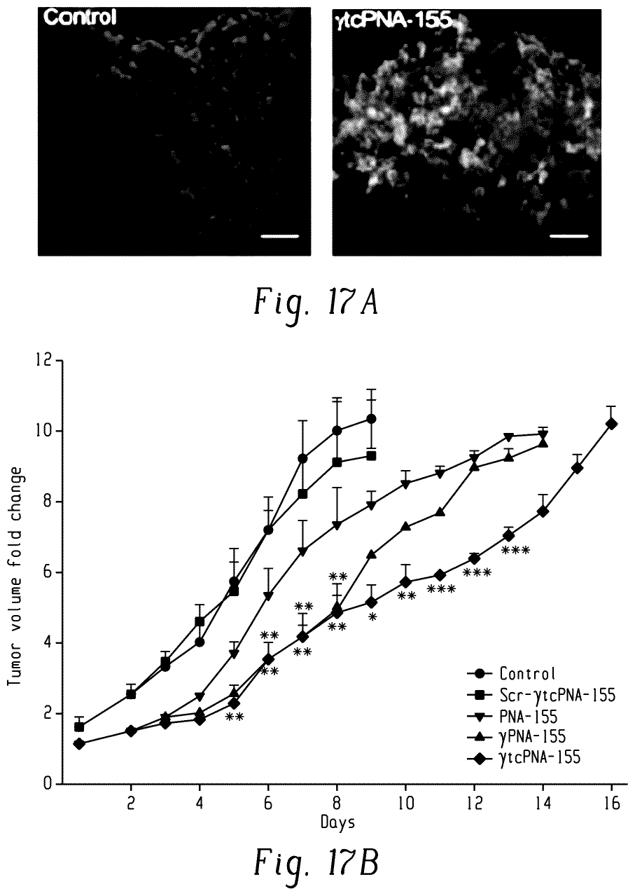

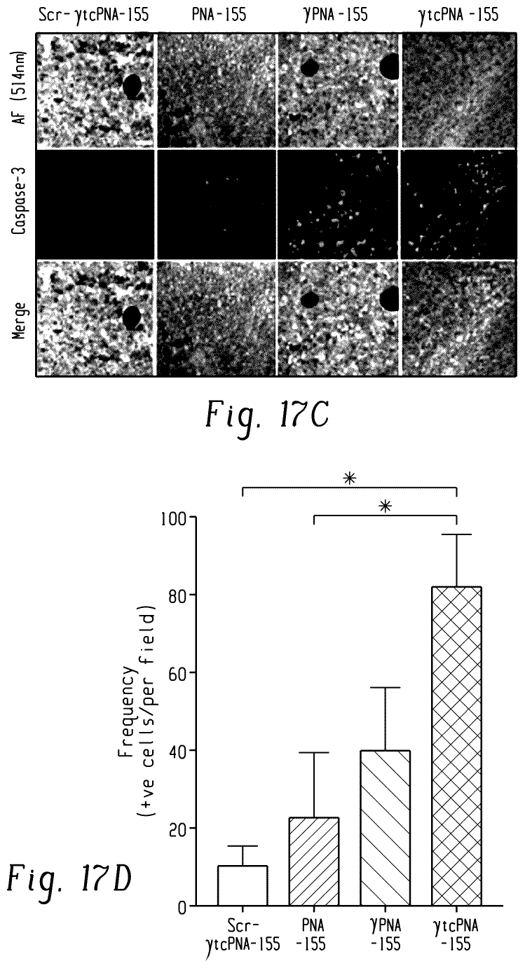

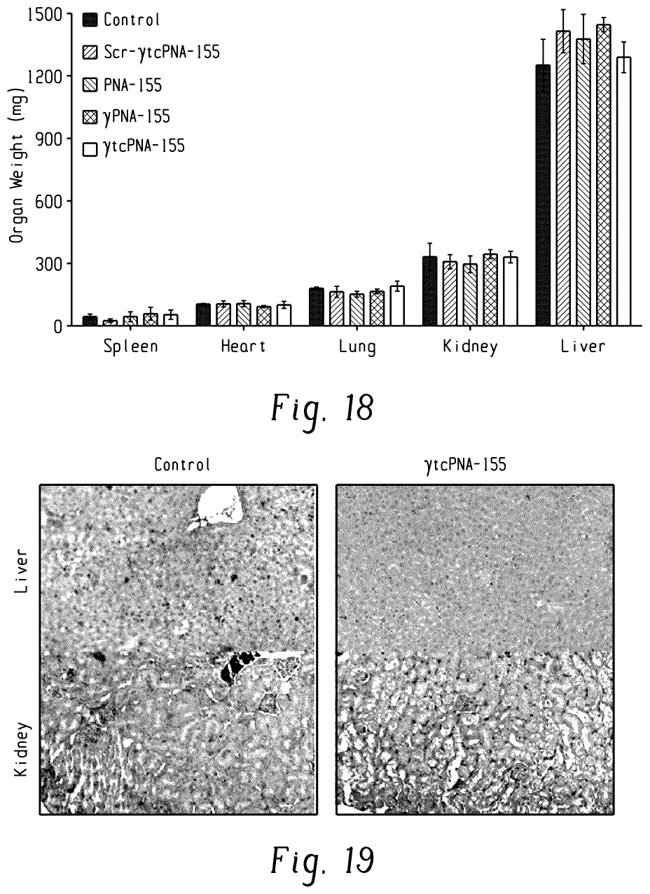

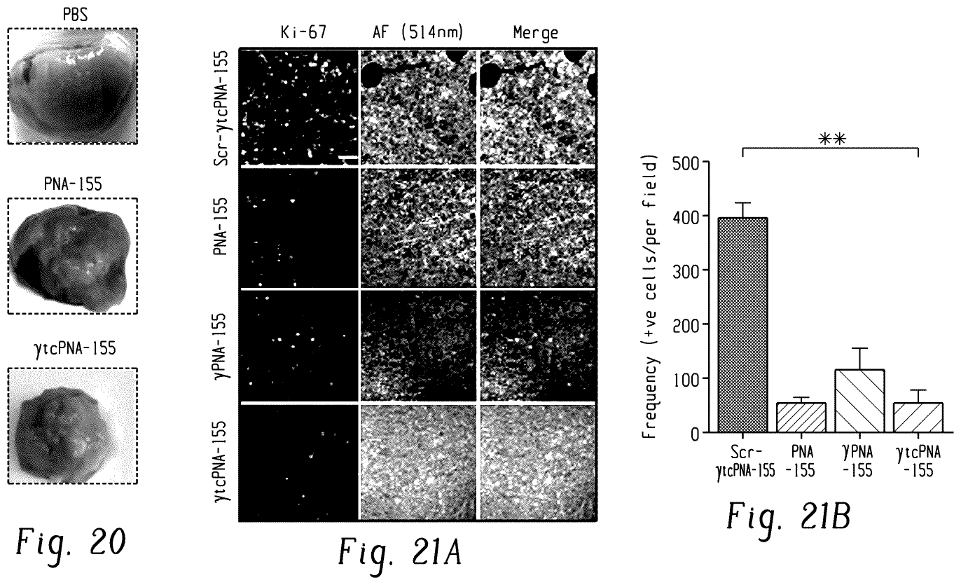

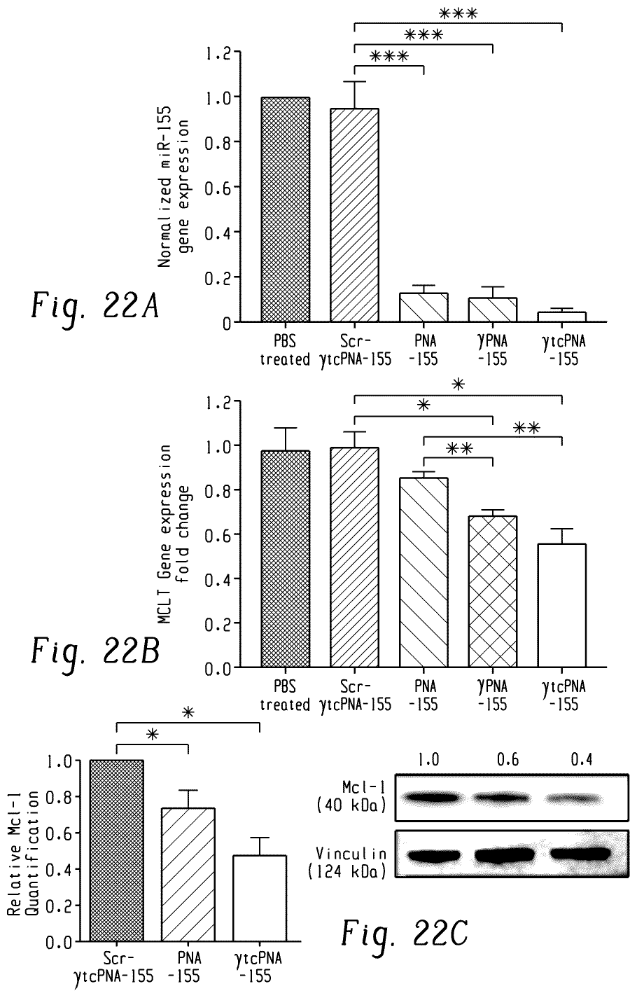

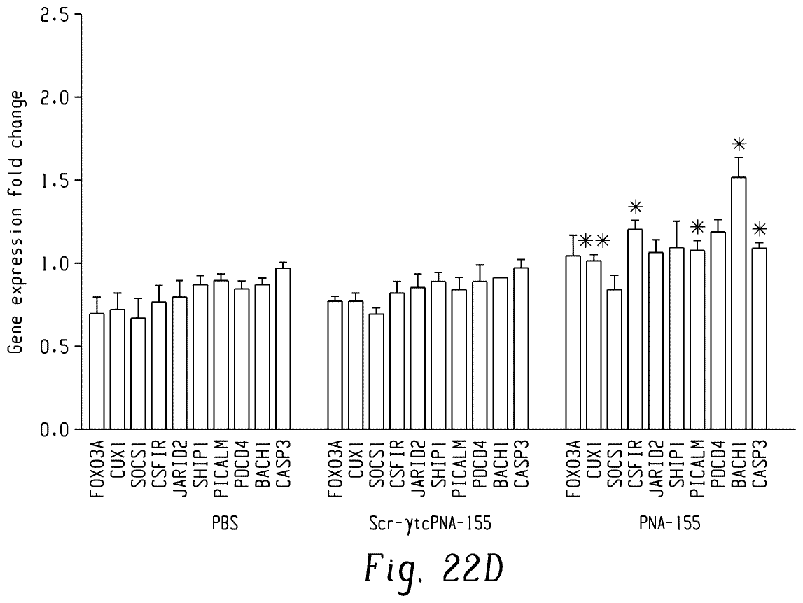

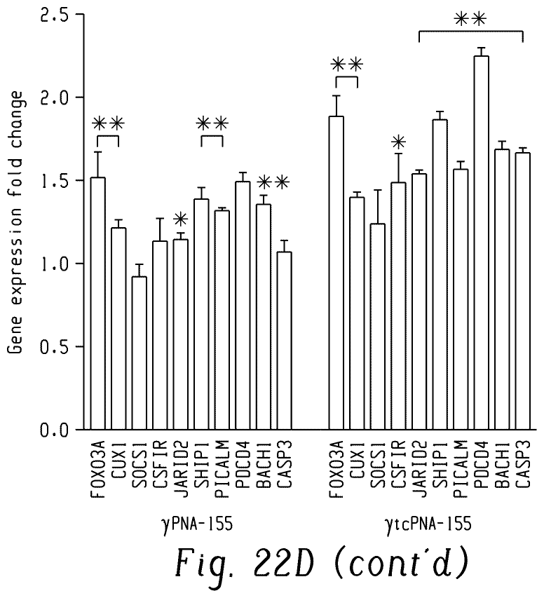

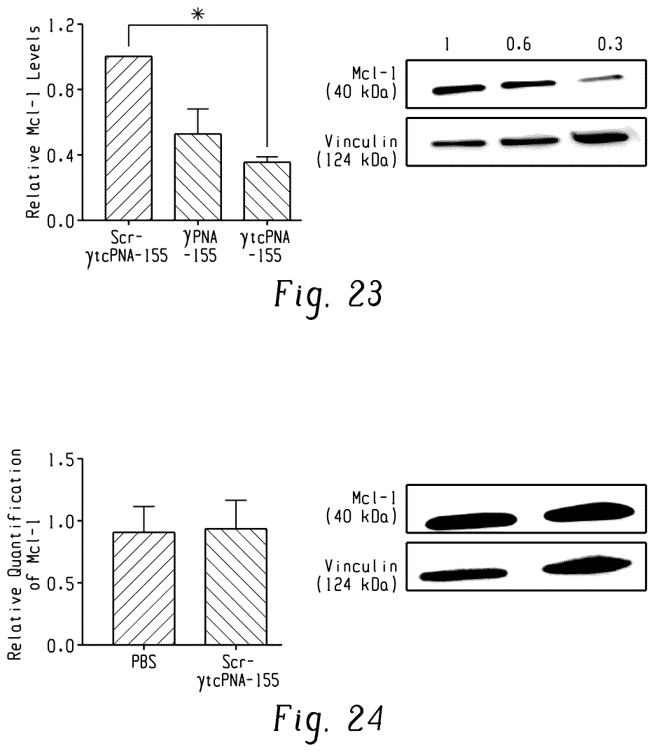

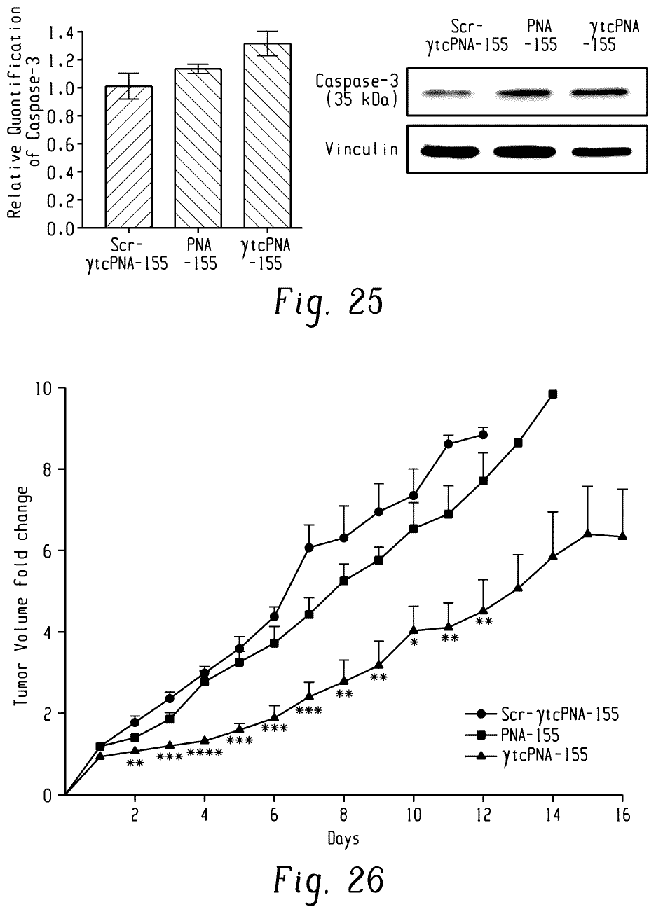

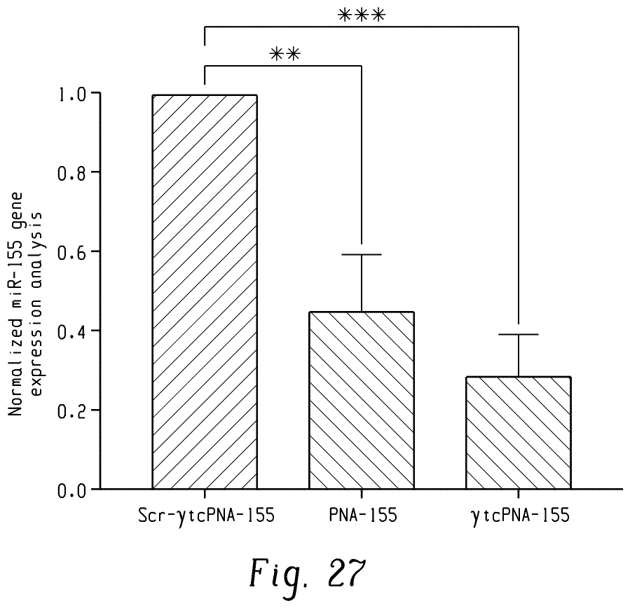

We designed and synthesized anti-miR-155 gamma tcPNAs (γtcPNA) and control PNA oligomers to test in a series of lymphoma cell lines and a xenograft mouse model (FIG. 1). It has been established that tail clamp PNAs can only bind to target RNA sequences containing homopurine stretches. Homopurine stretches are present at the 5′ end of the miR-155 sequence (FIG. 1B). Hence, we designed an anti-miR-155 gtcPNA containing Watson-Crick and Hoogsteen domain joined by flexible trioxo (—OOO—) linker to target the miR-155 sequence (FIGS. 1B & C, γtcPNA-155). In prior studies, it has been noted that gamma-modified PNAs exhibit superior binding and biocompatibility features compared to the conventional PNAs due to their right-handed helical pre-organization properties. gtcPNA-155 contains pseudoisocytosine (J) units on the Hoogsteen end as J can form hydrogen bonding at physiological pH. We also conjugated a TAMRA (5-Carboxytetramethylrhodamine) fluorophore to the 5′ terminal of γtcPNA-155 to study cellular uptake in cell lines and intra-tumoral biodistribution in the xenograft mice (FIG. 1C, γtcPNA-155-Tam). For comparison, we synthesized full-length regular PNA (23mer long) as it has been demonstrated to inhibit miR-155 in prior studies (FIG. 1C, PNA-155). We synthesized single-stranded full-length serine gamma PNAs that can bind to target miR-155 by Watson-Crick base pairing (FIG. 1C, γPNA-155) and a scrambled γtcPNA-155 (FIG. 1C, Scr-γtcPNA-155) as control. For cell permeability, we appended two arginine residues to the C- and N-terminus of each PNA. We selected four arginine residues to minimize the cytotoxicity associated with cationic domains. The PNAs, γPNAs and γtcPNAs were synthesized by established solid-phase synthesis based protocols, and quality control analyses were performed by reverse-phase high-performance liquid chromatography (RP-HPLC) (FIG. 2).

### Example 2: In Vitro Binding Studies

Next, we evaluated the binding affinity of γtcPNA-155 and PNA-155 with the miR-155 target by polyacrylamide gel electrophoresis (PAGE) based protocols (FIG. 1D). We incubated PNA-155 and γtcPNA-155 with miR-155 at indicated concentrations in physiological buffer and temperature and assessed the binding by PAGE followed by SYBR gold staining. As expected, we noticed an increase in the formation of a retarded band (PNA-155-miR-155 bound fraction) with the increase in concentration of PNA-155. We also noted the complete disappearance of the unbound miR-155 target at a miR-155: PNA-155 stoichiometry ratio of 1:2.0 (FIG. 1D, left panel). Whereas in the case of γtcPNA-155, we noticed the complete disappearance of unbound miR-155 target at miR-155: γtcPNA-155 stoichiometry ratio of 1:1.0 (FIG. 1D, right panel). These results confirmed that γtcPNA-155 possesses a stronger binding affinity compared to regular PNA-155. We also observed three distinct retarded band patterns in the case of γtcPNA-155-incubated with target miR-155. Three separate retarded bands are consistent with tcPNA binding modes with the complementary target strands. The top retarded band corresponds to the presence of γtcPNA-155 Watson-Crick, and the Hoogsteen domain bonded to miR-155 target. The other two retarded bands indicate binding with either Watson-Crick or Hoogsteen domain of γtcPNA-155 with the miR-155 target.

### Example 3: Cellular Uptake and Efficacy Studies in Lymphoma Cell Lines

Various studies established that miR-155 is upregulated in B-cell malignancies and is recognized as a therapeutically bona fide molecular target for treating lymphoma. Hence, we performed cellular uptake studies of γtcPNA-155-Tam in lymphoma cell lines (U2932 and SUDHL-2) by flow cytometry analysis. Significant cellular uptake of the TAMRA was observed in the U2932 (FIG. 3A) and SUDHL-2 cells (FIG. 4A) 24 hours' post-incubation with γtcPNA-155-TAMRA without using any transfection agent. Further, we also confirmed the cellular uptake of γtcPNA-155-TAMRA in U2932 cells by confocal microscopy (FIG. 4B).

Next, we assessed the miR-155 inhibitory activity of γtcPNA-155, γPNA-155 and PNA-155 in the U2932 cell line. U2932 cells were treated with a 500 nM dose of Scr-γtcPNA-155, PNA-155, γPNA-155 and γtcPNA-155 for 48 hours. Our RT-PCR results confirmed approximately 80%, 83% and 90% miR-155 inhibition with PNA-155, γPNA-155 and γtcPNA-155 treatments, respectively relative to scrambled control (FIG. 3B).

To verify miR-155 inhibition by the antimiRs, we evaluated the gene expression of known miR-155 downstream genes by RT-PCR-based gene expression analysis. High miR-155 gene expression results in repression of tumor suppressor genes via activation of the PI3-AKT signaling pathway, thereby promoting tumor cell survival, proliferation, and progression. Therefore, downregulating miR-155 will result in the de-repression of tumor suppressor genes. We examined the gene expression of panel of known miR-155 associated tumor suppressor genes by RT-PCR analysis after treatment with γtcPNA-155, γPNA-155 and PNA-155 (FIG. 3C). We noted significant de-repression of miR-155 target tumor suppressor genes in the U2932 cells treated with γPNA-155 and γtcPNA-155. In particular, CSF1R, CUX1, and SHIP1 negatively regulate PI3-AKT signaling and are a direct target of miR-155. We noted a 1.8-fold increase in CSF1R levels, a 1.6-fold increase in CUX1 levels and a 1.4-fold increase in the SHIP1 levels in γtcPNA-155 treated groups in comparison to PNA-155. Overall, we noted that γtcPNA-155 followed by γPNA-155 shows optimal de-repression of tumor suppressor genes in comparison to PNA-155. One plausible explanation of these findings is that γtcPNA-155 has a higher binding affinity with the miR-155 target.

In addition to the tumor suppressors, miR-155 is also known to impact expression of oncogenes. MCL1 is a known miR-155 downstream oncogene. Our gene expression results confirmed a 40% reduction in MCL1 gene expression levels after pre-treatment with γtcPNA-155 (FIG. 3D). γPNA-155 and PNA-155 pre-treatment results in 19% and 10% decline in MCL1 gene expression, respectively. We confirmed the effect of γtcPNA-155 on the validated miR-155 downstream genes MCL1 and Caspase-3 (CASP3) by western blot analysis. Caspase-3 has also been identified as a one of the direct targets of miR-155. Our results indicated that U2932 pretreatment with γtcPNA-155 at 500 nM concentration led to significant downregulation of Mcl-1 (50%) (FIGS. 3E and 5A) and upregulation of Caspase-3 (80%) (FIGS. 3F and 5B).

Next, we assessed the efficacy of γtcPNA-155 in SUDHL-2 cell lines that exhibit overexpression of miR-155. Consistent with our aforementioned findings, RT-PCR results show that pre-treatment of SUDHL-2 cell lines with γtcPNA-155 results in a 63% decrease in miR-155 gene expression (FIG. 6). We also investigated that the pre-treatment of SUDHL-2 cell lines with γtcPNA-155 results in significant upregulation of miR-155 associated tumor suppressor genes in comparison to Scr-γtcPNA-155 and PNA-155 (FIG. 7).

### Example 4: Reduction in Tumor Cell Viability after AntimiR Treatment

Prior studies indicated that miR-155 inhibition results in a decrease in cell proliferation and viability. Therefore, we assessed the dose-dependent cell viability in multiple lymphoma cell lines, U2932, SUDHL-5, and SUDHL-2 by trypan blue assay. As expected, we noticed a dose-dependent reduction in cell viability in U2932, SUDHL-2, and SUDHL-5 cell lines. We observed ˜80% decrease in cell viability in three cell lines at a 4UM concentration (FIG. 8). We also noted that γtcPNA-155 pretreatment causes a significant reduction in cell viability in U2932, SUDHL-5 and SUDHL-2 cell lines at a lesser concentration of 500 nM followed by γPNA-155 compared to the PNA-155 treated group (FIG. 8 and FIG. 9). These results are consistent with prior results signifying that γtcPNA-5 has a superior binding affinity followed by γPNA-155, and thus anti-miR-155 activity compared to regular full-length PNA-155. We did not notice any effect on cell viability with Scr-γtcPNA-155 in comparison to PBS treated control cells (FIG. 10).

Furthermore, to test whether the decrease in cell viability is due to apoptosis, we treated the U2932 cells with the same amount of γtcPNA-155 and PNA-155 and performed an annexin V-based apoptosis assay. Our results indicated that treatment with γtcPNA-155 results in increased apoptosis in U2932 cells than the PNA-155 and γPNA-155 treated group (FIG. 11). We also performed confocal imaging on U2932 cells treated with indicated PNAs and assessed the apoptosis by Annexin-V FITC stained methods. Consistent with our flow cytometry-based results, we noted higher apoptosis in the γtcPNA-155 treated cells (FIG. 12).

Prior studies successfully confirmed that pre-treatment of cobomarsen at a dose of 10 uM results in 4-fold increase in apoptosis in the U2932 lymphoma cell line compared to control. In a parallel comparison, we also performed an apoptosis assay after pre-treatment of U2932 cells with γtcPNA-155 at a dose of 10 uM. Our results indicated that the γtcPNA-155 result in a 4.5-fold increase in apoptosis in comparison to control (FIG. 13). Hence, we suggest that γtcPNA-155 efficacy is comparable to cobomarsen in a cell culture-based analysis.

Overall, these results demonstrate that γtcPNA-155, followed by γPNA-155, led to robust inhibition of miR-155, decreased cell viability, and increased apoptosis in U2932 cell lines. We also evaluated the safety of PNA-155, and γtcPNA-155 in PBMC cell lines in a dose-dependent manner using trypan blue assay. We did not notice a significant reduction in viability of PBMC cell lines indicating that the γtcPNA-155 and Scr-γtcPNA-155 do not cause any non-specific effects in the primary cells (FIG. 14).

### Example 5: γtcPNAs Suppresses Tumor Growth In Vivo

To evaluate if γtcPNA-155 can inhibit tumor growth more effectively than regular PNA-155 in vivo, we performed studies in xenografts derived from DLBCL cells. DLBCL U2932 cells were selected for implants as they show maximum miR-155 gene expression levels. Hence, they provide a robust xenograft model to study miR-155 inhibitory effects of γtcPNA-155. For our in vivo study, we assessed the anti-miR-155 impact of γtcPNA-155 and γPNA-155 compared to PNA-155 via intra-tumoral delivery. Cobomarsen, an investigative drug to inhibit miR-155 has also received orphan drug designation to treat lymphoma by intra-tumoral delivery. Since we selected the intra-tumoral route of delivery, we injected the U2932 cells in the right and left flank of mice (FIG. 15). After 10-14 days, when the tumor volume reached 100-200 mm3, the mice were divided into the five treatment groups. We also tested scrambled PNA (FIG. 1C, Scr-γtcPNA-155) as a control for in vivo study. The mice were randomized into groups based on the tumor volumes for uniform distribution of tumor volumes in each group. Before the efficacy study, we performed a biodistribution analysis of TAMRA-conjugated γtcPNA-155 (FIG. 1C, γtcPNA-155-Tam) at a dose of 3 mgkg⁻¹ intra-tumoral in the xenograft. We noticed a significant biodistribution in tumor after intra-tumoral injection by IVIS imaging (FIG. 16). We confirmed the biodistribution by confocal imaging of cryosections from the TAMRA treated group and control mice. Significant biodistribution of TAMRA fluorescence was noted in the tumor as compared to the control group (FIG. 17A).

For the efficacy study, each mouse received three intra-tumoral injections of either γtcPNA-155, γPNA-155 or PNA-155 on days 0, 7, 14. We noticed that the control and scrambled treated tumors reached 2000 mm³ much faster during our study, so we could not inject a third time in these mice. We did not observe: a significant decrease in the body weight (FIG. 18); histological damage to the kidney and liver (FIG. 19); general behavioral change in mice treated with indicated PNAs. Intra-tumoral administration reduced the tumor growth in the γtcPNA-155 treated group, followed by γPNA-155 and PNA-155 (FIGS. 17B and 20). In contrast, as stated earlier, there was no effect on the control and scrambled PNA control on the tumor volume.

To ascertain the efficacy of γtcPNA-155, the histological sections of tumors isolated from γtcPNA-155 treated mice were immunostained for apoptosis marker Caspase-3 (FIGS. 17C and 17D) and cell proliferation marker Ki67 (FIG. 20). We noted a significant decrease in the Ki67 positive cells and an increase in the Caspase-3 positive cells in the tumor sections isolated from γtcPNA-155 treated groups, which further corroborates the anti-tumor efficacy of γtcPNA-155.

We next assessed the levels of miR-155 and its downstream genes in the tumors of xenograft mice to assess the mechanism of tumor reduction. After tumor harvest, mouse and DLBCL cells were separated by a bead separation method to remove the false background from mice tissues. Subsequently, we investigated the level of miR-155 gene expression. Our results indicated that the γtcPNA-155 treated group shows a 90% decrease in miR-155 gene expression, whereas PNA-155 and γPNA-155 treated tumors indicated a 75% and 80% decline in the miR-155 expression respectively (FIG. 22A). As anticipated, we did not observe a change in the miR-155 expression level in PBS treated cells and scrambled groups.

Further, we measured the expression of miR-155 downstream genes, both tumor suppressor and oncogenes, as described in the in vitro result section. Upon γtcPNA-155 treatment, there was a significant reduction in the oncogenes and de-repression of tumor suppressor genes compared to the PNA-155 treated group. Compared to PNA-155 treated group, we observed about a 50% decrease in MCL1 mRNA levels in the γtcPNA-155 treated group (FIG. 22B). Further, we confirmed these results by measuring reduction in the protein levels of Mcl-1 in γtcPNA-155 treated tumors (FIG. 22C and FIG. 23). Consistent with tumor growth studies, we did not notice any alteration in the gene expression and Mcl-1 protein level for the Scr-γtcPNA-155 treated groups (FIG. 24). We also examined the in vivo de-repression of tumor suppressor genes as mentioned in the cell culture studies. Consistent with our cell culture-based results, we noted significant upregulation of tumor suppressor genes in the tumors of mice that received γtcPNA-155 intratumorally (FIG. 22D). We also measured Caspase-3 protein levels in the tumors and observed a 23% increase in the in vivo γtcPNA-155-treated tumors than Scr-γtcPNA-155-treated tumors (FIG. 25). These results collectively indicated that γtcPNA-155 exhibits a superior anti-miR-155 effect, followed by γPNA-155, decreasing tumor growth compared to its scrambled control and classical PNA-155.

Further, we corroborated our results in the SUDHL-2 derived xenograft mouse model. We injected the SUDHL-2 cells in the right and left flank of mice. After 14-21 days, when the tumor volume reached 100-200 mm3, the mice were randomized into three treatment groups based on uniform tumor volume distribution in each group. Consistent with prior in vivo results, we noted that γtcPNA-155 reduced the tumor growth as compared to the Scr-γtcPNA-155 and PNA-155 control (FIG. 26). We further confirmed that PNA-155 significantly reduces miR-155 expression level in the SUDHL-2 cell line-based xenograft after in vivo treatment. (FIG. 27)

### DISCUSSION

The antisense field has seen a rapid surge of FDA approvals of various nucleic acid-based drugs; Nusinersen, Onpattro®, Gilvaari®, Milasen to name a few, targeting coding mRNA for diverse therapeutic applications. However, targeting non-coding RNAs like miRNAs for clinical applications lags behind and still needs further optimization as a broader platform. miRNAs have been established for their key roles in cancer progression and transformation. Especially, miR-155 is considered an important biomarker and highly expressed in B-cell lymphoma and DLBCL. Hence, in recent years, interest and research in this area has grown. miRagen Therapeutics (now Viridian Therapeutics) has made strides in developing cobomarsen as a drug candidate to target miR-155 for cutaneous T-cell lymphoma treatment. Cobomarsen has shown promise in decreasing miR-155 levels followed by reduced tumor burden. Recent studies also indicated that systemic delivery of cobomarsen shows favorable outcomes in patients that developed resistance to CHOP and CAR-T-cell therapy. Overall, these studies underpin the significance of miR-155 as an important molecular target for developing precision medicine for lymphoma therapy.

However, practicing miRNA therapeutics has been challenging due to delivery, plasma stability, and moderate efficacy of nucleic acid analogs-based antimiRs. Novel chemical modifications have been performed to increase the efficacy and enzymatic stability of next-generation antimiRs with minimal off-target effects. Though numerous synthetic nucleic acid analogs have been developed as potential antimiRs, LNA and PNA have gained enormous attention due to their enzymatic stability and superior physico-biochemical properties. Both full-length and seed-targeting LNAs have been explored as potential anti-miR-155 based therapeutics. Though LNA-based cobomarsen has shown promise, the field still needs to increase the antimiR-based repertoire that can be utilized for broader applications and targeting of diverse miRNAs with increased efficacy.

PNA has been widely used as antimiR for targeting full-length miRNA. Apart from its binding properties, PNA-based technology has been amenable to several delivery platforms like nanoparticles, liposomes, and peptide conjugations, to target the tumor microenvironment and inhibit the target miRNA selectively. Conventional regular PNA targets full-length miRNAs and inhibits their target mRNA interaction by steric hindrance. Herein, we tested novel gamma modified tcPNA antimiRs that can bind with both Watson-Crick and Hoogsteen base pairing with a miR-155 target containing a homopurine stretch and inhibit its activity. In prior studies, it has been well-established that gamma tcPNAs can induce a higher percentage of gene editing than the regular PNA due to their high binding affinity. We report that gamma tcPNAs can inhibit the miR-155 at a higher level than conventional full-length PNAs.

Herein, we also noted that full-length serine-gamma PNA causes increased miR-155 inhibition in both cell culture and in vivo analysis compared to conventional PNA-155. Prior studies indicated that poly-lactic-co-glycolic acid (PLGA) nanoparticles loaded with diethylene-glycol units containing gamma anti-miR-210 PNA results in significant miR-210 inhibition in a HeLa cell line derived xenograft. Diethylene-glycol containing gamma PNAs have enhanced loading and release in the PLGA NPs as compared to the regular PNA-210, resulting in increased antimiR efficacy. To the best of our knowledge, our study here is the first where a parallel comparison of the antimiR efficacy of cationic serine gamma PNAs and conventional PNAs based on their binding affinity has been performed. In addition, serine gamma PNAs have not been explored before for in vitro and in vivo antimiR efficacy.

We performed an extensive analysis of gamma tcPNA's anti-miR-155 activity in both cell culture and in vivo analysis. We validated our anti-miR-155 results by gene expression and protein level analysis of direct and indirect miR-155 targets in U2932 lymphoma cell lines. We demonstrated that repressing miR-155 levels decreases cell viability in multiple lymphoma cell lines SUDHL-2, SUDHL-5, and U2932. We also found that gamma tcPNA-155 induced apoptosis in vitro. Our in vivo results in the xenograft mice indicated that gamma tcPNA design could inhibit miR-155 and further effect the miR-155 downstream targets in vivo more efficiently compared to regular PNA-155. Further, a decrease in cellular proliferation and an increase in apoptosis markers mechanistically supports the retardation of tumor growth in mice receiving intra-tumoral injections of gamma tcPNA-155. Prior studies successfully demonstrated that pre-treatment of cobomarsen at a dose of 10 μM results in 4-fold increase in apoptosis in the U2932 lymphoma cell line compared to control. Our results indicate that treatment of U2932 cell lines with gamma tcPNAs at a dose of 10 μM causes a 4.5-fold increase in apoptosis compared to control. Hence, we examined that gamma tcPNA results are comparable to cobomarsen based on in-vitro studies. Various important miR-155 targets are involved in lymphoma cell proliferation. In a prior study, it has been noted that CUX1 and WEE1 are established miR-155 predicted targets and play an essential role in tumor proliferation. In both in vitro and in vivo analysis, we noted that gene expression of multiple miR-155 targets, including CUX1 and WEE1, increased after treatment with gamma tcPNA-155.

We have presented a novel antimiR strategy that could target multiple miRNA-based molecular targets for diverse therapeutic applications.

In conclusion, an efficient antimiR-based repertoire is required to target the miRNAs that are involved in disease pathogenesis. Our results demonstrate that gamma-modified tail-clamp-PNA-based anti-miR-155 not only improves its affinity toward the target miR-155 but also increases its efficacy in retarding tumor growth. Further, gamma-modified tail-clamp-PNA-based antimiRs appeared safe in our mice throughout treatment. Lastly, the gamma tail-clamp PNA technology can also be employed to target mRNAs and other non-coding RNAs involved in the development of other health disorders.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A peptide nucleic acid (PNA) oligomer that forms a PNA/RNA/PNA triplex structure, wherein the PNA oligomer has the formula:
5′-first PNA segment-flexible linker-second PNA segment-3′
wherein
the first PNA segment is complementary to a homopurine stretch in the ribonucleic acid (RNA),
the second PNA segment is complementary to a region of the RNA including the homopurine stretch,
the first PNA segment and the second PNA segment form the PNA/RNA/PNA triplex structure with the RNA, and
the RNA is a coding or noncoding RNA.

2. The PNA oligomer of claim 1, wherein
the first PNA segment, the second PNA segment, or both the first PNA segment and the second PNA segment comprises one or more gamma monomers;
the first PNA segment, the second PNA segment, or both the first PNA segment and the second PNA segment are serine gamma modified; or
a combination thereof.

3. (canceled)

4. The PNA oligomer of claim 1, wherein the first PNA segment comprises 3-10 pyrimidines.

5. The PNA oligomer of claim 1, wherein the first PNA segment comprises one more pseudoisocytosine units.

6. The PNA oligomer of claim 1, wherein the first PNA segment comprises only pseudoisocytosine units and thymidine units.

7. The PNA oligomer of claim 1, wherein the flexible linker comprises 1 to 10 units of 8-amino-3,6-dioxaoctanoic acid, 6-aminohexanoic acid, 8-amino-2, 6, 10-trioxaoctanoic acid, 11-amino-3, 6, 9-trioxaundecanoic acid, or a combination thereof.

8. The PNA oligomer of claim 1, further comprising one or more arginine residues appended at a carboxyl-terminus (C-terminus), an amino-terminus (N-terminus), or both the C-terminus and the N-terminus of the PNA oligomer.

9. The PNA oligomer of claim 1, wherein the RNA is microRNA (miRNA), messenger RNA (mRNA), or long noncoding RNA.

10. The PNA oligomer of claim 9, wherein the miRNA is miR-155.

11. The PNA oligomer of claim 10, wherein the second PNA segment is complementary to miR-155 and comprises the sequence CCCCTATCACGATTAGCATTAA (SEQ ID NO:1).

12. The PNA oligomer of claim 9, wherein the second PNA segment is complementary to any of the miRNA sequences of SEQ ID NOs: 8-28.

13. The PNA oligomer of claim 1, further comprising a detectable label.

14. A composition comprising the PNA oligomer of claim 1, and a pharmaceutically acceptable excipient.

15. A method for reducing expression of a targeted RNA involved in health disorders in a subject, the method comprising
providing to a cell of the subject in vivo or ex vivo a PNA oligomer according to claim 1, wherein the binding of the PNA oligomer to the targeted RNA reduces expression of the targeted RNA.

16. The method of claim 15, wherein the health disorder is a B-cell malignancy and wherein the targeted RNA is miR-155.

17. The method of claim 16, wherein
the second PNA segment of the PNA oligomer is complementary to miR-155 RNA and has the sequence CCCCTATCACGATTAGCATTAA (SEQ ID NO:6);
the B-cell malignancy is Diffuse Large B-Cell Lymphoma (DLBCL); or
a combination thereof.

18. (canceled)

19. A method for increasing gene expression of an miR-155 associated tumor suppressor gene, the method comprising downregulating miR-155 expression by providing to a cell a PNA oligomer of claim 1, wherein the tumor suppressor gene is any of colony stimulating factor 1 receptor (CSF1R), cut like homeobox 1 (CUX1), and Src homology 2 (SH2) domain-containing inositol polyphosphate 5-phosphatase 1 (SHIP1).

20. A method for reducing expression of miR-155 associated oncogenes, the method comprising downregulating miR-155 expression in a cell comprising providing to the cell a PNA oligomer of claim 1, wherein the miR-155 associated oncogene is Myeloid cell leukemia-1 (MCL1).

21. A method for increasing gene expression of miR-155 associated oncogenes, the method comprising downregulating miR-155 expression by providing to a cell a PNA oligomer of claim 1, wherein the miR-155 associated oncogene is Caspase-3.

22. A method for increasing apoptosis of tumor cells overexpressing miR-155, the method comprising downregulating miR-155 expression by providing to a cell a PNA oligomer of claim 1.


SEQUENCE LISTING (28 sequences)

SEQ ID NO: 1 - DNA, 23 bp, synthetic construct
  source 1..23: mol_type = other DNA; note = PNA-155 sequence
  misc_feature 1: note = ArgArg covalently linked to backbone
  misc_feature 23: note = ArgArg covalently linked to backbone
  modified_base 1..23: mod_base = OTHER; note = peptide nucleic acid
acccctatcacgattagcattaa

SEQ ID NO: 2 - RNA, 23 nt, Homo sapiens
  source 1..23: mol_type = genomic RNA
ttaatgctaatcgtgataggggt

SEQ ID NO: 3 - DNA, 22 bp, Synthetic construct
  source 1..22: mol_type = other DNA; note = gammatcPNA-155
  misc_feature 22: note = Arg-Arg covalently linked to the backbone
  modified_base 1..22: mod_base = OTHER; note = peptide nucleic acid
  misc_feature 1: note = Arg-Arg-thymine-pseudoisocytosine-pseudoisocytosine -pseudoisocytosine-pseudoisocytosine-11-amino-3,6,9-trioxa undecanoic acid covalently linked to the backbone
cccctatcacgattagcattaa

SEQ ID NO: 4 - DNA, 21 bp, Synthetic construct
  source 1..21: mol_type = other DNA; note = scramble sequence
  misc_feature 1: note = Arg-Arg-adenine-thymine-pseudoisocytosine-thymine -adenine-11-amino-3,6,9-trioxaundecanoic acid covalently linked to the backbone
  modified_base 3: mod_base = OTHER; note = n is pseudoisocytosine
  modified_base 11: mod_base = OTHER; note = n is pseudoisocytosine
  misc_feature 21: note = Arg-Arg covalently linked to the backbone
  modified_base 19: mod_base = OTHER; note = n is pseudoisocytosine
  modified_base 1..21: mod_base = OTHER; note = peptide nucleic acid
acntgccattncacgaacnct

SEQ ID NO: 5 - DNA, 23 bp, Synthetic construct
  source 1..23: mol_type = other DNA; note = anti-miR-155 PNA oligomer
acccctatcacgattagcattaa

SEQ ID NO: 6 - DNA, 22 bp, Synthetic construct
  source 1..22: mol_type = other DNA; note = anti-miR-155 PNA oligomer
cccctatcacgattagcattaa

SEQ ID NO: 7 - DNA, 22 bp, Synthetic construct
  source 1..22: mol_type = other DNA
  modified_base 1..22: mod_base = OTHER; note = peptide nucleic acid
  misc_feature 1: note = Thymine-pseudoisocytosine-pseudoisocytosine-pseudoi socytosine-pseudoisocytosine-11-amino-3,6,9-trioxaundecano ic acid covalently linked to the backbone
cccctatcacgattagcattaa

SEQ ID NO: 8 - RNA, 22 nt, Homo sapiens
  source 1..22: mol_type = genomic RNA
tagcttatcagactgatgttga

SEQ ID NO: 9 - RNA, 22 nt, Homo sapiens
  source 1..22: mol_type = genomic RNA
acctggcatacaatgtagattt

SEQ ID NO: 10 - RNA, 21 nt, Homo sapiens
  source 1..21: mol_type = genomic RNA
tactcaaaaagctgtcagtca

SEQ ID NO: 11 - RNA, 24 nt, Homo sapiens
  source 1..24: mol_type = genomic RNA
ttaatgctaatcgtgataggggtt

SEQ ID NO: 12 - RNA, 23 nt, Homo sapiens
  source 1..23: mol_type = genomic RNA
taccctgtagaaccgaatttgtg

SEQ ID NO: 13 - RNA, 22 nt, Homo sapiens
  source 1..22: mol_type = genomic RNA
tggaaacatttctgcacaaact

SEQ ID NO: 14 - RNA, 22 nt, Homo sapiens
  source 1..22: mol_type = genomic RNA
tggagtgtgacaatggtgtttg

SEQ ID NO: 15 - RNA, 22 nt, Homo sapiens
  source 1..22: mol_type = genomic RNA
agttcttcagtggcaagcttta

SEQ ID NO: 16 - RNA, 22 nt, Homo sapiens
  source 1..22: mol_type = genomic RNA
aacccgtagatccgatcttgtg

SEQ ID NO: 17 - RNA, 23 nt, Homo sapiens
  source 1..23: mol_type = genomic RNA
cggggccgtagcactgtctgaga

SEQ ID NO: 18 - RNA, 24 nt, Homo sapiens
  source 1..24: mol_type = genomic RNA
tttggcaatggtagaactcacact

SEQ ID NO: 19 - RNA, 22 nt, Homo sapiens
  source 1..22: mol_type = genomic RNA
acctggcatacaatgtagattt

SEQ ID NO: 20 - RNA, 22 nt, Homo sapiens
  source 1..22: mol_type = genomic RNA
ctcagtagccagtgtagatcct

SEQ ID NO: 21 - RNA, 16 nt, Homo sapiens
  source 1..16: mol_type = genomic RNA
ctaggaggccttggcc

SEQ ID NO: 22 - RNA, 23 nt, Homo sapiens
  source 1..23: mol_type = genomic RNA
taaagtgcttatagtgcaggtag

SEQ ID NO: 23 - RNA, 22 nt, Homo sapiens
  source 1..22: mol_type = genomic RNA
tccctgagaccctaacttgtga

SEQ ID NO: 24 - RNA, 23 nt, Homo sapiens
  source 1..23: mol_type = genomic RNA
caaagtgcttacagtgcaggtag

SEQ ID NO: 25 - RNA, 21 nt, Homo sapiens
  source 1..21: mol_type = genomic RNA
cataaagtagaaagcactact

SEQ ID NO: 26 - RNA, 23 nt, Homo sapiens
  source 1..23: mol_type = genomic RNA
aacattcaacgctgtcggtgagt

SEQ ID NO: 27 - RNA, 22 nt, Homo sapiens
  source 1..22: mol_type = genomic RNA
ggctacaacacaggacccgggc

SEQ ID NO: 28 - RNA, 22 nt, Homo sapiens
  source 1..22: mol_type = genomic RNA
tgtaaacatcctcgactggaag